(19) United States
(12) Patent Application Publication
(10) Pub. No.: US 2013/0225477 A1
(43) Pub. Date: Aug. 29, 2013

(54) Oral Lactoferrin in the Treatment of Severe Sepsis

(71) Applicants: Atul Varadhachary, Bellaire, TX (US); Karel Petrak, Houston, TX (US)

(72) Inventors: Atul Varadhachary, Bellaire, TX (US); Karel Petrak, Houston, TX (US)

(73) Assignee: AGENNIX AG, Heidelberg (DE)

(21) Appl. No.: 13/580,434

(22) PCT Filed: Feb. 25, 2011
(86) PCT No.: PCT/EP2011/052831

§ 371 Date: Nov. 20, 2012

Related U.S. Application Data
(60) Provisional application No. 61/307,944, filed on Feb. 25, 2010.

(51) Int. Cl. A61K 38/40

(52) U.S. Cl. CPC A61K 38/40
USPC 514/1.4

(57) ABSTRACT

The present invention relates to lactoferrin for use in the treatment of severe sepsis. In particular, the present invention relates to methods of effectively treating sepsis, in particular, severe sepsis, by administering orally a composition of lactoferrin (LF). More particularly, the present invention relates to methods of treating prophylactically or therapeutically sepsis, in particular, severe sepsis, by administering orally a composition of lactoferrin to patients with an APACHE II score ≦25, in particular <25.

The present invention relates to lactoferrin for use in the treatment of severe sepsis. In particular, the present invention relates to methods of effectively treating sepsis, in particular, severe sepsis, by administering orally a composition of lactoferrin (LF). More particularly, the present invention relates to methods of treating prophylactically or therapeutically sepsis, in particular, severe sepsis, by administering orally a composition of lactoferrin to patients with an APACHE II score ≦25, in particular <25.

Sepsis is defined as the Systemic Inflammatory Response Syndrome (SIRS) to an infective process. Sepsis is a result of a bacterial infection that can originate anywhere in the body. Common sites are the genitourinary tract, the liver or biliary tract, the gastrointestinal tract, and the lungs. Less common sites are intravenous lines, surgical wounds, decubitus ulcers and bedsores. The infection is usually confirmed by a positive blood culture. The infection can lead to a shock, called septic shock. Septic shock is more often caused by hospital-acquired gram-negative bacilli and usually occurs in immuno-compromised patients and those with chronic diseases. In about ⅓ of patients, however, it is caused by gram-positive cocci and by Candida organisms. The diagnosis of sepsis is based on the presence of at least two out of the following four criteria: tachycardia (heart rate >90 bpm), hyperventilation (respiratory frequency >20/min or pCO₂ₑₓₚ <35 mm Hg), fever (>38.3° C.) or hypothermia (<36° C.) and leukocytosis (>12,000/μL) or leukopenia (<4,000/μL).

There are about 750,000 cases of sepsis in the U.S.A. every year, at least 225,000 of which are fatal. Only one drug has been approved for sepsis so far—a recombinant human activated protein C that exhibits antithrombotic, anti-inflammatory and profibrinolytic properties.

Sepsis is the clinical syndrome defined by the presence of both infection (either known or suspected) and a systemic inflammatory response. Infection is suspected when physiologic manifestations present, such as white blood cells (WBCs) noted in a normally sterile body fluid, perforated viscus, chest radiograph consistent with pneumonia, or a clinical syndrome associated with a high likelihood of infection (eg, ascending cholangitis). Evidence of a systemic inflammatory response includes derangement in vital signs and WBC count.

Physiologically, sepsis is characterized by cytokine release, increased expression of adhesion molecules, release of reactive oxygen species, and expression of acute-phase proteins. Both local and systemic sequelae are observed following the ischemia-reperfusion injury to the gut that is observed in sepsis. The resulting increases in gut permeability are associated with increased bacterial translocation which can further exacerbate the septic state.

Severe sepsis is defined as sepsis plus one or more organ dysfunctions (e.g., as determined by SOFA [Sepsis-related Organ Failure Assessment] score) and, in particular, one or more organ dysfunctions selected from

acute lung injury

coagulation abnormalities

thrombocytopenia

altered mental status

renal failure

liver failure

cardiac failure or/and

hypoperfusion with lactic acidosis

The SOFA Score is outlined in more detail below

See SOFA Score³
Sepsis-related Organ Failure (SOFA) Score
European Society of Intensive Care Medicine (ESICM), 1994
SOFA score evaluate status of the following organ systems separately:
1. Respiration 2. Coagulation 3. Liver 4. Cardiovascular
5. Central Nervous System 6. Renal

| | | SOFA Score | | | | |
|---|---|---|---|---|---|---|
| Organ System | Measurement | 0 | 1 | 2 | 3 | 4 |
| Respiration | PaO₂/FiO₂, mmHg | Normal | <400 | <300 | <200 | <100 |
| Coagulation | Platelets × 10³/mm³ | Normal | <150 | <100 | <50 | <20 |
| Liver | Bilirubin, mg/dL | Normal | 1.2-1.9 | 2.0-5.9 | 6.0-11.9 | >12.0 |
| Cardiovascular | Hypotension | Normal | MAP <70 mHg | Dopamine ≦5 | Dopamine >5 | Dopamine >15 |
| | | | | or dobutamine | or epinephrine | or epinephrine |
| | | | | (any dose)* | ≦0.1 or | >0.1 or |
| | | | | | norepinephrine ≦0.1 | norepinephrine >0.1 |
| Central Nervous System | Glasgow Coma | Normal | 13-14 | 10-12 | 6-9 | <6 |
| | Score | | | | | |
| Renal | Creatinine, mg/dL | Normal | 1.2-1.9 | 2.0-3.4 | 3.5-4.9 | >5.0 |
| | or | | | | | |
| | Urine output | | | | <500 mL/d | <200 mL/d |

*adrenergic agents administered for at least 1 hour (doses given are in mcg/kg/min)
See, Vincent JL, Moreno R, Takata J, Willatts S, De Mendonça A, Bruining H, Reinhart CK, Suter PM, and Thijs LG. The SOFA (Sepsis-related Organ Failure Assessment) score to describe organ dysfunction/failure. On behalf of the Working Group on Sepsis-Related Problems of the European Society of Intensive Care Medicine. Intensive Care Med. 1996; 22: 707-710.

An organ dysfunction of one of the organ systems is present when the SOFA Score is 1, in particular, when the SOFA Score is 2, preferably when the SOFA Score is 3, and more preferably when the SOFA Score is 4.

For the category of sepsis cases which are designated “severe sepsis” current treatment options are very limited. Primarily, treatment focuses on support for distressed organ functions (heart, lungs and kidneys), breathing support, and fluid therapy (Dellinger R P, Levy M M, Carlet J M, et al., Surviving Sepsis Campaign: International guidelines for management of severe sepsis and septic shock. Intensive Care Medicine (2008) 34:17-60 and Crit Care Med 2008; 36(1) 296-327). In particular, available pharmaceutical therapeutics for severe sepsis are limited. Antibiotics are used to treat the underlying infection, however severe sepsis is not resolved by antibiotic therapy alone. Corticosteroids and vasopressors are used to control hypotension from septic shock in some cases.

In the case of severe sepsis, a distinction can be made between patients having a documented infection (i.e. documented severe sepsis) or a diagnosis without microbiological documentation (i.e. culture-negative severe sepsis). The 28-day mortality is about 56% in patients with documented severe sepsis and about 60% in patients having culture-negative severe sepsis.

The current severe sepsis treatment consists of eradicating the underlying infection and providing supportive care of any associated organ dysfunction. The treatment includes identification of pathogen, eliminating the source of infection, fluid therapy (hemodynamic support), empiric antimicrobial therapy, inotropic and vasoactive drug support and pulmonary support (oxygen, mechanical ventilation).

The current understanding of sepsis suggests that new treatments should be developed that prevent and/or neutralize cytokine release, inhibit inflammation and coagulation and facilitate fibrinolysis.

In November 2001, FDA approved the first biologic treatment for severe sepsis. This drug, Drotrecogin Alfa (Activated) (Xigris®), is a genetically engineered form of a naturally occurring human protein, activated Protein C. Its beneficial effects on mortality and morbidity in severe sepsis have been attributed to its anti-inflammatory, anticoagulant, and profibrinolytic effects. Drotrecogin Alfa (Activated) improves patient mortality rates but has limitations and, in particular, significant side effects. Drotrecogin Alfa (Activated) causes serious bleeding in a significant number of patients which can be fatal. Drotrecogin Alfa (Activated) failed to demonstrate efficacy in pediatric populations (18 years or less of age). Drotrecogin Alfa (Activated) does not have a therapeutic effect for severe sepsis patients having an APACHE II score of ≦25 (Abraham, Edward, et al., the Administration of Drotrecogin Alfa (Activated) in Early Stage Severe Sepsis (ADDRESS) Study Group, Drotrecogin Alfa (Activated) for Adults with Severe Sepsis and a Low Risk of Death N Engl J Med 2005 353: 1332-1341). Finally, in some subpopulations such as sepsis patients with no more than one organ dysfunction or APACHE II scores of ≦25, Drotrecogin Alfa (Activated) appears to actually increase mortality rates.

A pharmaceutical under investigation for severe sepsis is eritoran tetrasodium (ACCESS: A Controlled Comparison of Eritoran Tetrasodium and Placebo in Patients With Severe Sepsis (NCT00334828), Phase III clinical trial (ongoing). Phase II clinical trial results for eritoran tetrasodium indicate efficacy for severe sepsis with APACHE II scores. Tidswell, M, et al., Phase 2 trial of eritoran tetrasodium (E5564), a toll-like receptor 4 antagonist, in patients with severe sepsis. Crit Care Med. 2010 January; 38(1):72-83). However, it is stated “For the subjects in the lowest quartile of APACHE II scores (less than 21), mortality rate was higher in the eritoran tetrasodium 105-mg treated group (12.0% vs. 0.0% placebo, CMH chi-square test, p=0.083”. Thus, severe sepsis cases continue to present an unmet medical need, particularly those cases where patients have lower APACHE II scores and/or exactly one organ dysfunction.

The pharmaceutical preparation containing recombinant human activated protein C, Drotrecogin Alfa (Activated), which is e.g. marketed as Xigris®, however, has substantial side effects. Therefore, this medicament is admitted only in the case of APACHE II scores >25 and, further, cannot be applied to children.

The Acute Physiology and Chronic Health Evaluation (APACHE) II scoring system is a severity-of-disease classification system which uses a point score based upon initial values of 12 routine physiologic measurements, age, and previous health status to provide a general measure of severity of disease, resulting in a score ranging from 0-71 that correlates with the risk of hospital death (Knaus et al, 1985); 71 indicating highest risk of death. The physiological measurements include: body temperature, heart rate, blood pressure, mean arterial pressure, respiratory rate, pulmonary function, white blood cell count, arterial pH, blood oxygenation and serum levels of creatinine, sodium, potassium and HCO₃. The APACHE II score can be used to determine the severity of sepsis.

The APACHE II prognostic system is explained below (Knaus W A, Wagner D P, Draper E A, Zimmerman J E, Bergner M, Bastos P G, Sirio C A, Murphy D J, Lotring T, Damiano A: The APACHE II prognostic system. Risk prediction of hospital mortality for critically ill hospitalized adults. Chest 1991, 100:1619-1636).

APACHE II Scoring System
A Acute Physiology Points:

| | High Abnormal Range | | | | | Low Abnormal Range | | | |
|---|---|---|---|---|---|---|---|---|---|
| Physiologic variable | +4 | +3 | +2 | +1 | 0 | +1 | +2 | +3 | +4 |
| Temperature (rectal, ° C.) | ≧41 | 39-40.9 | | 38.5-38.9 | 36-38.4 | 34-35.9 | 32-33.9 | 30-31.9 | ≦29.9 |
| Mean Arterial Pressure (mmHg) | ≧160 | 130-155 | 110-129 | | 70-109 | | 50-69 | | ≦49 |
| Heart rate (ventricular response) | ≧180 | 140-179 | 110-139 | | 70-109 | | 55-69 | 40-54 | ≦39 |
| Respiratory rate (non-ventilated | ≧50 | 35-49 | | 25-34 | 12-24 | 10-11 | 6-9 | | ≦5 |
| orientation) | | | | | | | | | |
| Oxygenation: AaDO₂ or PaO₂ | | | | | | | | | |
| (mmHg) | | | | | | | | | |
| a. FIO₂ ≧0.5 record only AaDO | ≧500 | 350-499 | 200-349 | | <200 | | | | |
| b. FIO₂ <0.5 record only PaO₂ | | | | | PO₂ >70 | PO₂ 61-70 | | PO₂ 55-60 | PO₂ <55 |
| Arterial pH | ≧7.7 | 7.6-7.69 | | 7.5-7.59 | 7.33-7.49 | | 7.25-7.32 | 7.15-7.24 | <7.15 |
| Serum sodium (mMd/L) | ≧180 | 160-179 | 155-159 | 150-154 | 130-149 | | 120-129 | 111-119 | ≦110 |
| Serum potassium (mMd/L) | ≧7 | 6-6.9 | | 5.5-5.9 | 3.5-5.4 | 3-3.4 | 2.5-2.9 | | <2.5 |
| Serum creatinine (mg/100 mL) | ≧3.5 | 2-3.4 | 1.5-1.9 | | 0.6-1.4 | | <0.6 | | |
| (double point score for acute renal | | | | | | | | | |
| failure) | | | | | | | | | |
| Hcmatocrit (%) | ≧60 | | 50-59.9 | 46-49.9 | 30-45.9 | | 20-29.9 | | <20 |
| White Blood Count | ≧40 | | 20-39.9 | 15-19.9 | 3-14.9 | | 1-2.9 | | <1 |
| Glasgow Coma Score (GCS) | | | | | | | | | |
| Score = 15 minutes actual GCS | | | | | | | | | |
| Total Acute Physiology Score | | | | | | | | | |
| Serum HCO₃ (venus, mMd/L) | ≧52 | 41-51.9 | | 32-40.9 | 22-31.9 | | 18-21.9 | 15-17.9 | <15 |
| (non preferred, use if no ABGs) | | | | | | | | | |

| | | |
|---|---|---|
| B AGE POINTS: | | |
| Assign points to | C CHRONIC HEALTH POINTS: | |
| age as follows: | If the patient has a history of severe organ insufficiency or is | APACHE II SCORE |

| Age (yrs) | Points | immunocompromised, assign points as follows: | Sum of A + B + C |
|---|---|---|---|
| ≧44 | 0 | a. nonoperative or emergency past-operative patients: 5 points | A APS Points |
| 45-54 | 2 | b. elective postoperative patients: 2 points | B Age Points |
| 56-64 | 3 | Definitions: Organ insufficiency or immunocompromised state evident | C Chronic Health Points |
| 65-74 | 5 | prior to this hospital admission and conforming to the following criteria: | TOTAL APACHE II |
| ≦75 | 6 | LIVER: Biopsy proven cirrhosis and documented portal hypertension; | |
| | | episodes of past upper GI bleeding attributed to portal hypertension; or | |
| | | prior episodes of hepatic failure/encephalopathy/coma. | |
| | | CARDIOVASCULAR: New York Heart Association Class IV. | |
| | | RESPIRATORY: Chronic restrictive, obstructive, or vascular disease | |
| | | resulting in severe exercise restriction, ie, unable to climb stairs or | |
| | | perform household duties; or documented chronic hypoxia, hypercapnia, | |
| | | secondary polyeythemia, severe pulmonary hypertension (>40 mmHg), | |
| | | or respirator dependency. | |
| | | RENAL: Receiving chronic dialysis. | |
| | | IMMUNOCOMPROMISED: Patient has received therapy that | |
| | | suppresses resistance to infection, eg. immunosuppression, chemotherpy, | |
| | | radiation, long term or recent high dose steroids, or has a disease that is | |
| | | sufficiently advanced to suppress resistance to infection (eg, leukemia, | |
| | | lymphoma, AIDS) | |

The pathogenesis of septic shock resulting from bacteremia and sepsis (SIRS) is not completely understood. The bacterial toxins generated by the infecting organisms trigger complex immunologic reactions. A large number of mediators including tumour necrosis factor, leukotriene, lipoxygenase, histamine, bradykinin, serotonin, and interleukin-2 have been implicated in addition to endotoxin (the lipid fraction of the lipopolysaccharides released from the cell wall of gram-negative enteric bacilli). Initially, vasodilation of arteries and arterioles occurs, decreasing peripheral arterial resistance with normal or increased cardiac output even though the ejection fraction may be decreased when heart rate increases. Later, cardiac output may decrease and peripheral resistance may increase. Despite increased cardiac output, blood flow to the capillary exchange vessels is impaired causing eventually failure of one or more of the visceral organs.

In experimental animals, for example in mice injected with endotoxin, endotoxemia and endotoxin-induced death accompanied by the oxidative burst and overproduction of inflammatory mediators occurs. Intraperitonially administered lactoferrin has been described to play a role in the pathogenesis of endotoxemia, primarily through binding to the bacterial endotoxins (Kruzel M L et al., Clin Exp Immunol 2002; 130:25-31). Other effects of parenteral lactoferrin have also been described for example, intraperitoneal administration of lactoferrin 1 hour before lipopolysaccharide (LPS) challenge resulted in an inhibition of several mediators, namely TNF-alpha by 82%, IL-6 by 43%, IL-10 by 47% at 2 hours following LPS injection, and reduction in nitric oxide (NO) (by 80%) at 6 hours post-shock. Prophylactic i.p. administration of lactoferrin at 18 hours prior to LPS injection resulted in similar decreases in TNF-alpha (95%) and in NO (62%). Similarly, when lactoferrin was administered i.p. as a therapeutic post-induction of endotoxic shock, significant reductions were apparent in TNF-alpha and NO in serum.

It has been reported in the literature that oral lactoferrin is not absorbed systemically through the mature gut to any significant degree (Heyman M and Desjeux J-F, Significance of intestinal food protein transport. J Pediatr Gastroenterol Nutr 1992; 15:48-57; Fransson G B, Thoren-Tolling K, Jones B, Hambraeus L, and Lonnerdal B. Absorption of lactoferrin-iron in suckling pigs. Nutr Res 1983; 3:373-84; Holloway N M, Lakritz J, Tyler J W, Carlson S L. Serum lactoferrin concentrations in colostrum-fed calves. Am J Vet Res 2002 April; 63(4):476-8) and the literature also assumes that a large part of lactoferrin's role is related to the binding of systemically circulating endotoxins. For example, a 2-phase GLP pharmacokinetic study was conducted in the Rhesus monkey to determine the oral availability of rhLF. Standard dosing volume of 4 mL/kg was administered by oral gavage. A comparison was made to the pharmacokinetics of i.v.-administered rhLF. The oral dose of rhLF was 1000 mg/kg. Following this oral dose, the plasma concentrations of rhLF were not significantly higher than the pre-dose, endogenous lactoferrin values. The calculated absolute bioavailability was less than 0.5%.

Lactoferrin is a single chain metal binding glycoprotein. Many cells types, such as monocytes, macrophages, lymphocytes, and brush-border cells, are known to have lactoferrin receptors. In addition to lactoferrin being an essential growth factor for both B and T lymphocytes, lactoferrin has a wide array of functions related to host primary defense mechanisms. For example, lactoferrin has been reported to activate natural killer (NK) cells, induce colony-stimulating activity, activate polymorphonuclear neutrophils (PMN), regulate granulopoeisis, enhance antibody-dependent cell cytotoxicity, stimulate lymphokine-activated killer (LAK) cell activity, and potentiate macrophage toxicity.

It was an object of the invention to provide a treatment for severe sepsis.

According to the invention, this object is achieved by lactoferrin for use in the treatment of severe sepsis.

The present invention is directed to lactoferrin for use in the treatment of severe sepsis. In particular, the present invention is directed to a method for treating prophylatically or therapeutically severe sepsis. Further, septic shock or related conditions such as multiple organ failure and acute respiratory distress syndrome (ARDS) can be treated with lactoferrin.

According to the invention it was found that severe sepsis can be successfully treated with lactoferrin. Severe sepsis is defined as sepsis plus one or more organ dysfunctions. The present invention is the first to use a lactoferrin composition and, in particular, an oral lactoferrin composition as an effective treatment for severe sepsis and, in particular, to patients with an APACHE II score ≦25, preferably <25.

In a clinical study it has been found that lactoferrin can be used in the treatment of severe sepsis. In particular, it was found that when using lactoferrin a higher survival rate is achieved. Further, relatively few occurrences of side effects are observed with lactoferrin.

Lactoferrin is especially suitable for treatment of severe sepsis, i.e. sepsis plus one or more organ dysfunctions, in particular, one or more organ dysfunctions selected from acute lung injury, coagulation abnormalities, thrombocytopenia, altered mental status, renal failure, liver failure, cardiac failure or/and hypoperfusion with lactic acidosis. Such organ failure is present when the SOFA Score is 1, in particular, when the SOFA Score is 2, preferably when the SOFA score is 3 and, more preferably, when the SOFA Score is 4.

Severe sepsis is the case, in particular, when, besides sepsis, at least one acute organ dysfunction is present which is due to sepsis that is newly developed and not explained by other disease processes or the effects of treatment. Such acute organ dysfunctions are, in particular, defined as follows:
Cardiovascular—the requirement for vasopressors*, in addition to adequate fluid resuscitation**, to maintain a mean arterial blood pressure (MAP) >65 mm Hg or a systolic blood pressure >90 mm Hg. *Vasopressors are defined here as dopamine at ≧5 mcg/kg/min or any dose of norepinephrine, epinephrine, phenylephrine, or vasopressin with the intent to support blood pressure. Dobutamine and dopexamine are not considered vasopressors.**Adequate fluid resuscitation is defined here as one of the following: i) a minimum of a 20 mL/kg (ideal body weight) intravenous fluid challenge (crystalloid or equivalent colloid), or ii) if measured, a central venous pressure (CVP)≧8 mm Hg, or iii) a pulmonary artery occlusion pressure (PAOP)≧12 mm Hg.
Respiratory—Pa₂/FiO₂ ratio ≦250, or ≦200 if the lung is the primary site of infection, and, if measured, a pulmonary capillary wedge pressure not suggestive of central venous volume overload
Renal—i) an absolute increase in serum creatinine of ≦0.3 mg/dL from baseline, or ii) a ≧50% increase in serum creatinine from baseline, or iii) a urine output <0.5 mL/kg/hr for ≧1 hour. The preceding criteria must be met despite adequate fluid resuscitation** (defined as above for cardiovascular dysfunction)
Hematologic—platelet count <80,000/mm³ or decreased by ≧50% from the highest value recorded within 3 days prior to randomization
Metabolic—serum lactate >4.0 mm/L (or equivalent in other units)

Further, it has been found that lactoferrin according to the invention has favorable effects also and especially in the case of patients whose general condition is still reasonably good, i.e. patients particularly having a baseline APACHE II score of ≦25, in particular, <25, preferably <21, more preferably <20. Due to lactoferrin having relatively few side effects, it can be administered also to patients whose general condition is still relatively good. There has been no approved drug so far for those patients.

According to the invention, lactoferrin can be used for treating severe sepsis, wherein the severe sepsis comprises at least one organ dysfunction. Severe sepsis can comprise several organ dysfunctions, in particular, up to eight organ dysfunctions, preferably up to six organ dysfunctions, more preferably five organ dysfunctions, even more preferably four organ dysfunctions, in particular, three organ dysfunctions and, more preferably two organ dysfunctions. Most preferably, lactoferrin is used for the treatment of severe sepsis, wherein exactly one organ dysfunction occurred.

It was also found in the conducted clinical studies that lactoferrin acts especially favorably against severe sepsis in patients having unimpaired cardiovascular functions. Thus, lactoferrin can be used especially favorably in the case of patients having no organ dysfunction with respect to cardiovascular functions.

The lactoferrin used according to the invention can be basically any lactoferrin, e.g. human or bovine lactoferrin. It is preferred to use human lactoferrin. Preferably, complete lactoferrin, also referred to as talactoferrin, is used.

Lactoferrin is preferably provided in a composition which can be administered enterally, in particular, orally.

Administration is preferably effected in an amount of from 1.5 mg to 100 g doses every 8 hours, more preferably, in an amount of from 1.0 to 5 g doses every 8 hours.

Further, it has been found that lactoferrin can be used successfully also in the case of children, i.e. in the case of patients being less than 18 years old.

Lactoferrin has been previously identified as a potential therapeutic of sepsis (U.S. Pat. App. 2004/0152624). Lactoferrin is disclosed as being useful for treating the underlying infection (bacteremia), septic shock and certain organ dysfunctions (e.g. lung impairment or Acute Respiratory Distress Syndrome). Each of these may or may not exist in specific severe sepsis cases (Morrell M R, Micek S T, Kollef M H. Infect, The management of severe sepsis and septic shock. Dis Clin North Am. 2009 September; 23(3):485-501). Septic shock for example would be infrequent in low APACHE II scores (less than 25, 21 or 20) category patients. Thus while lactoferrin is disclosed as having utility in sepsis therapy generically (i.e. to treat the underlying bacteremia) and for specific symptoms such as septic shock, lactoferrin has not been discussed as a therapeutic for patients classified as having severe sepsis nor for such patients having low APACHE II scores. Because severe sepsis is a notoriously difficult subcategory of sepsis, the general disclosure of lactoferrin for, e.g., bacteremia therapy, could not be reasonably extrapolated to any expected improvement in outcomes in severe sepsis cases. The inventors have surprisingly found that human lactoferrin does improve mortality and other outcome measures in severe sepsis cases. Even more surprisingly, this improvement is seen in low APACHE II scores patients where other interventions discussed above are ineffective or possibly even increase mortality rates over placebo.

The following numbered sentences more readily define the invention as described herein.
1. A method of treating severe sepsis comprising the step of administering to a subject a lactoferrin composition in an amount sufficient to provide an improvement in the severe sepsis of said subject.
2. A method of treating severe sepsis comprising the step of supplementing the mucosal immune system in a subject by increasing the amount of lactoferrin in the gastrointestinal tract.
3. A method of decreasing mortality of a subject having severe sepsis comprising the step of administering to said subject a lactoferrin composition in an amount sufficient to a attenuate the severe sepsis to decrease mortality of said subject.
4. The method of sentences 1 to 3, wherein the improvement is attenuating severe sepsis.
5. The method of sentences 1 to 4, wherein the improvement is attenuating at least one organ failure.
6. The method of sentences 1 to 5, wherein the improvement is a decrease in morbidity of said subject.
7. The method of sentences 1 to 5, wherein the improvement is a decrease in mortality of said subject.
8. The method of sentences 1 to 7, wherein said lactoferrin composition is dispersed in a pharmaceutically acceptable carrier.
9. The method of sentences 1 to 8, wherein said lactoferrin is mammalian lactoferrin.
10. The method of sentences 1 to 9, wherein said lactoferrin is human or bovine.
11. The method of sentences 1 to 10, wherein said lactoferrin is recombinant lactoferrin.
12. The method of sentences 1 to 11 further comprising administering an antacid in conjunction with said lactoferrin composition.
13. The method of sentences 1 to 12, wherein the amount of the lactoferrin that is administered is about 1 mg to about 300 g per day, preferably about 3 mg to about 100 g per day and, in particular, 3 g to about 20 g per day.
14. The method of sentences 1 to 13 further comprising administering a metal chelator, preferably dispersed in a pharmaceutically acceptable carrier.
15. The method of sentence 14, wherein the metal chelator is ethylenediaminetetraacetic acid (EDTA) or ethylenebis(oxyethylenenitrilo)]tetraacetic acid (EGTA).
16. The method of sentence 14 or 15, wherein the amount of EDTA that is administered is about 0.01 μg to about 20 g per day.
17. The method of sentences 14 to 16, wherein the ratio of EDTA to lactoferrin in the composition that is administered is from 1:10,000 to about 2:1.
18. The method of sentences 1 to 17 further comprising administering the lactoferrin composition in combination with an antibiotic.
19. The method of sentences 1 to 18, wherein the lactoferrin is administered enterally.
20. The method of sentences 1 to 19, wherein the lactoferrin is administered orally.
21. The method of sentences 16 to 20, wherein said lactoferrin stimulates interleukin-18 in the gastrointestinal tract.
22. The method of sentences 1 to 21, wherein interleukin-18 stimulates the production or activity of immune cells.
23. The method of sentences 1 to 22, wherein said lactoferrin reduces the production or activity of pro-inflammatory cytokines.
24. The method of sentences 1 to 23, wherein said composition that is administered is a liquid formulation.
25. The method of sentences 1 to 24, wherein said composition that is administered is a solid formulation, in particular, with an enteric coating.
26. The method of sentences 1 to 25, wherein said composition that is administered is a solid formulation without an enteric coating.
27. The method of sentences 1 to 26, wherein oral administration is via a nasogastric tube.
28. The method of sentences 1 to 27, wherein the improvement is decreasing the levels of circulating bacteria.

For a more complete understanding of the present invention, the above-described pre-clinical data are shown graphically in the following Figures.

As used herein, the term “bactererima” as used herein is defined as having bacteria in the blood of the subject.

The term “sepsis” as used herein is defined as a Systemic Inflammatory Response Syndrome to an infective process in which severe derangement of the host immune system fails to prevent extensive ‘spill over’ of inflammatory mediators from a local infection focus into the systemic circulation. The diagnosis of sepsis is based on the presence of at least two out of the following four criteria: tachycardia (heart rate >90 bpm), hyperventilation (respiratory frequency >20/min or pCO₂ₑₓₚ <35 mm Hg), fever (>38.3° C.) or hypothermia (<36° C.) and leukocytosis (>12,000/μL) or leukopenia (<4,000/μL).

The term “septic shock” as used herein is a consequence of sepsis in which the systemic inflammatory response leads to the failure of vital organs' function (for example of the lungs as in ARDS). A significant feature of septic shock is that the failure of vital organ function has not occurred yet but is in progress and will occur within short.

The term “gram-negative bacteria” or “gram-negative bacterium” as used herein is defined as bacteria which have been classified by the Gram stain as having a red stain. Gram-negative bacteria have thin walled cell membranes consisting of a single layer of peptidoglycan and an outer layer of lipopolysacchacide, lipoprotein, and phospholipid. Exemplary organisms include, but are not limited to, Enterobacteriacea consisting of Escherichia, Shigella, Edwardsiella, Salmonella, Citrobacter, Klebsiella, Enterobacter, Hafnia, Serratia, Proteus, Morganella, Providencia, Yersinia, Erwinia, Buttlauxella, Cedecea, Ewingella, Kluyvera, Tatumella and Rahnella. Other exemplary gram-negative organisms not in the family Enterobacteriacea include, but are not limited to, Pseudomonas aeruginosa, Stenotrophomonas maltophilia, Burkholderia, Cepacia, Gardenerella, Vaginalis, and Acinetobacter species.

The term “gram-positive bacteria” or “gram-positive bacterium” as used herein refers to bacteria, which have been classified using the Gram stain as having a blue stain. Gram-positive bacteria have a thick cell membrane consisting of multiple layers of peptidoglycan and an outside layer of teichoic acid. Exemplary organisms include, but are not limited to, Staphylococcus aureus, coagulase-negative staphylococci, streptococci, enterococci, corynebacteria, and Bacillus species.

The term “antimicrobial” as used herein is defined as a substance that inhibits the growth of microorgansims without damage to the host, for example antibiotics, anti-fungal and antiseptics.

The term “antibiotics” as used herein is defined as a substance that inhibits the growth of microorganisms without damage to the host. For example, the antibiotic may inhibit cell wall synthesis, protein synthesis, nucleic acid synthesis, or alter cell membrane function. Classes of antibiotics that can possibly be used include, but are not limited to, macrolides (i.e., erythromycin), penicillins (i.e., nafcillin), cephalosporins (i.e., cefazolin), carbepenems (i.e., imipenem, aztreonam), other beta-lactam antibiotics, beta-lactam inhibitors (i.e., sulbactam), oxalines (i.e. linezolid), aminoglycosides (i.e., gentamicin), chloramphenicol, sufonamides (i.e., sulfamethoxazole), glycopeptides (i.e., vancomycin), quinolones (i.e., ciprofloxacin), tetracyclines (i.e., minocycline), fusidic acid, trimethoprim, metronidazole, clindamycin, mupirocin, rifamycins (i.e., rifampin), streptogramins (i.e., quinupristin and dalfopristin) lipoprotein (i.e., daptomycin), polyenes (i.e., amphotericin B), azoles (i.e., fluconazole), and echinocandins (i.e., caspofungin acetate).

The term “morbidity” as used herein is the state or condition of being diseased. Yet further, morbidity can also refer to the ratio of incidence, for example the number of sick subjects or cases of diseases in relationship to a specific population.

The term “mortality” as used herein is the state of being mortal or causing death. Yet further, mortality can also refer to the death rate or the ratio of number of deaths to a given population.

The term “oral administration” as used herein includes oral, buccal, enteral, rectal or intragastric administration.

The term “pharmaceutically acceptable carrier” as used herein includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

The term “lactoferrin” or “LF” as used herein refers to native or recombinant lactoferrin. Native lactoferrin can be obtained by purification from mammalian milk or colostrum or from other natural sources. Recombinant lactoferrin (rLF) can be made by recombinant expression or direct production in genetically altered animals, plants, fungi, bacteria, or other prokaryotic or eukaryotic species, or through chemical synthesis.

Lactoferrin is known to have anti-infective properties and anti-inflammatory properties, and has also been shown to restore and maintain the barrier properties of the GI mucosa. Native lactoferrin (LF) is an iron-binding protein found mainly in external secretions of mucosal epithelia such as breast milk, saliva, tears, bile, and pancreatic fluid. It is also found in plasma secreted from the secondary granules of neutrophils, which are the main source of LF in plasma as it is secreted upon neutrophil stimulation

In animal models of sepsis, enterally administered talactoferrin has been shown to protect against mortality induced by bacteria and endotoxin administration.

A preferably used lactoferrin is a human lactoferrin (talactoferrin alfa or TLF, also known as recombinant human lactoferrin or rhLF), produced in Aspergillus niger var. awamori. Talactoferrin is essentially equivalent (structurally and functionally) to native lactoferrin from human milk, as demonstrated by a comparison of the 3-dimensional structure, molecular weight, biological activity and other physicochemical properties, and differs only in the nature of glycosylation. Like the native protein, talactoferrin displays anti-infective and anti-inflammatory properties that have been demonstrated in in-vitro, preclinical and human clinical studies.

### Non-clinical Pharmacology of Talactoferrin

Talactoferrin has demonstrated anti-microbial (bactericidal and bacteriostatic), anti-inflammatory, anti-viral, anti-fungal, anti-tumor activity as well as activity in animal models of asthma and sepsis.

Oral TLF, which acts locally at the level of the intestinal enterocytes and the GALT, can help stabilize the gut and interrupt the cycle of damaging sepsis-related events. Talactoferrin has been observed to protect against gut damage in both preclinical and clinical studies, and to reduce translocation of bacteria across the gut.

The term “metal chelator” as used herein refers to a compound which binds metal. Metal chelators that can be used in the present invention include the divalent metal chelators, for example, ethylenediaminetetraacetic acid (EDTA), [ethylenebis (oxyethylenenitrilo)]tetraacetic acid (EGTA), 1,2-bis(2-aminophenoxy)ethane-N,N,N′,N′-tetraacetic acid (BAPTA), hydroxyethylethylene diamine triacetic acid, (HEDTA) or salts thereof.

The term “subject” or “patient” as used herein, is taken to mean any mammalian subject to which a lactoferrin composition is orally administered according to the methods described herein. A skilled artisan realizes that a mammalian subject, includes, but is not limited to humans, monkeys, horses, pigs, cows, dogs, cats, rats and mice. In a specific embodiment, the methods of the present invention are employed to treat a human subject.

The term “effective amount” or “therapeutically effective amount” as used herein refers to an amount that results in an improvement or remediation of the symptoms of the disease or condition.

The term “treating” and “treatment” as used herein refers to administering to a subject a therapeutically effective amount of a recombinant human lactoferrin composition so that the subject has an improvement in the disease. The improvement is any improvement or remediation of the symptoms associated with severe sepsis or its consequences. The improvement is an observable or measurable improvement. Thus, one of skill in the art realizes that a treatment may improve the disease condition, but may not be a complete cure for the disease.

The term “preventing” as used herein refers to minimizing, reducing or suppressing the risk of developing a disease state or parameters relating to the disease state or progression or other abnormal or deleterious conditions.

### A. PHARMACEUTICAL COMPOSITIONS

The present invention includes a composition comprising lactoferrin that is dispersed in a pharmaceutical carrier.

The lactoferrin used according to the present invention can be obtained through isolation and purification from natural sources, for example, but not limited to mammalian milk. The lactoferrin is preferably mammalian lactoferrin, such as bovine or human lactoferrin. In preferred embodiments, the lactoferrin is human lactoferrin produced recombinantly using genetic engineering techniques well known and used in the art, such as recombinant expression or direct production in genetically altered animals, plants or eukaryotes, or chemical synthesis. See, i.e., U.S. Pat. Nos. 5,571,896; 5,571,697 and 5,571,691, which are herein incorporated by reference.

The inventive treatment involves oral administration of a lactoferrin composition alone or in combination with a metal chelator.

Metal chelators that can be used in combination with lactoferrin, include the metal chelators, for example, ethylenediaminetetraacetic acid (EDTA), [ethylenebis(oxyethylenenitrilo)]tetraacetic acid (EGTA), 1,2-bis(2-aminophenoxy)ethane-N,N,N′,N′-tetraacetic acid (BAPTA), hydroxyethylethylene diamine triacetic acid, (HEDTA) or any salts thereof. More preferrably, EDTA is used in combination with lactoferrin.

Further, in accordance with the present invention, the inventive composition suitable for oral administration is provided in a pharmaceutically acceptable carrier with or without an inert diluent. The carrier should be assimilable or edible and includes liquid, semi-solid, i.e., pastes, or solid carriers. Except insofar as any conventional media, agent, diluent or carrier is detrimental to the recipient or to the therapeutic effectiveness of a lactoferrin preparation and/or the metal chelator contained therein, its use in an orally administrable lactoferrin and/or metal chelator for use in practicing the methods of the present invention is appropriate. Examples of carriers or diluents include fats, oils, water, saline solutions, lipids, liposomes, resins, binders, fillers and the like, or combinations thereof.

In accordance with the present invention, the composition is combined with the carrier in any convenient and practical manner, i.e., by solution, suspension, emulsification, admixture, encapsulation, microencapsulation, absorption and the like. Such procedures are routine for those skilled in the art.

In a specific embodiment of the present invention, the composition in powder form is combined or mixed thoroughly with a semi-solid or solid carrier. The mixing can be carried out in any convenient manner such as grinding. Stabilizing agents can be also added in the mixing process in order to protect the composition from loss of therapeutic activity through, i.e., denaturation in the stomach. Examples of stabilizers for use in an orally administrable composition include buffers, antagonists to the secretion of stomach acids, amino acids such as glycine and lysine, carbohydrates such as dextrose, mannose, galactose, fructose, lactose, sucrose, maltose, sorbitol, mannitol, etc., proteolytic enzyme inhibitors, and the like.

Further, the composition which is combined with a semi-solid or solid carrier can be further formulated into hard or soft shell gelatin capsules, tablets, or pills. More preferably, gelatin capsules, tablets, or pills are enterically coated. Enteric coatings prevent denaturation of the composition in the stomach or upper bowel where the pH is acidic. See, i.e., U.S. Pat. No. 5,629,001. Upon reaching the small intestines, the basic pH therein dissolves the coating and permits the composition to be released and absorbed by specialized cells, e.g., epithelial enterocytes and Peyer's patch M cells.

In another embodiment, a powdered composition is combined with a liquid carrier such as, e.g., water or a saline solution, with or without a stabilizing agent.

Upon formulation, solutions are administered in a manner compatible with the dosage formulation and in such amount as is therapeutically effective to result in an improvement or remediation of the symptoms. The formulations are easily administered in a variety of dosage forms such as ingestible solutions, drug-release capsules and the like. Some variation in dosage can occur depending on the condition of the subject being treated. The person responsible for administration can, in any event, determine the appropriate dose for the individual subject. Moreover, for human administration, preparations meet sterility, general safety and purity standards as required by FDA Office of Biologics standards.

### B. TREATMENT OF SEVERE SEPSIS

In accordance with the present invention, lactoferrin is used in the treatment of severe sepsis. One skilled in the art can determine the therapeutically effective amount of the composition to be administered to a subject based upon several considerations, such as local effects, pharmacodynamics, absorption, metabolism, method of delivery, age, weight, disease severity and response to the therapy. Typical amounts for administration are from about 1 mg to about 300 g per day, preferably about 3 mg to about 100 g per day, and, in particular, 3 g to about 20 g per day. Administration is effected preferably 3 times a day, for example, at doses of 0.5 to 10 g each, preferably 1 to 5 g, even more preferably 1.5 to 3 g per administration. Administration is preferably effected orally. Oral administration of the composition includes oral, buccal, enteral, rectal or intragastric administration.

In further embodiments, the composition is administered in conjunction with an antacid. Thus, an antacid is administered prior or substantially simultaneously with or after oral administration of the composition. The administration of an antacid just prior or immediately following the administration of the composition may help to reduce the degree of inactivation of the lactoferrin in the digestive tract. Examples of appropriate antacids include, but are not limited to, sodium bicarbonate, magnesium oxide, magnesium hydroxide, calcium carbonate, magnesium trisilicate, magnesium carbonate and aluminum hydroxide gel.

In a preferred embodiment of the present invention, the lactoferrin composition is administered in an effective amount to minimize the effects of severe sepsis. The amount of lactoferrin in the composition may vary from about 1 mg to about 100 g, in particular, from 10 mg to 50 g, preferably from 500 mg to 10 g. Preferably, the composition that is orally administered contains the range of 1 mg to 50 g of lactoferrin per day. In specific embodiments, the composition is given in a single dose or multiple doses. The single dose may be administered daily, or multiple times a day, or multiple times a week. In a further embodiment, the lactoferrin is given in a series of doses. The series of doses may be administered daily, or multiple times a day, weekly, or multiple times a week. In a further embodiment, the lactoferrin is given as a continuous infusion via a nasogastric tube. Preferably, lactoferrin is administered in doses three times per day.

More preferably, the composition of the present invention also contains metal chelators, for example, but not limited to ethylenediaminetetraacetic acid (EDTA), [ethylene-bis-(oxyethylenenitrilo)]tetraacetic acid (EGTA), 1,2-bis-(2-aminophenoxy)ethane-N,N,N′,N′-tetraacetic acid (BAPTA), hydroxyethylethylene diamine triacetic acid, (HEDTA) or salts thereof. The amount of the metal chelator in the composition may vary from about 0.01 μg to about 20 g. A preferred metal chelator is EDTA. More preferably, the composition that is orally administered contains the ratio of 1:10,000 to about 2:1 EDTA to lactoferrin.

Treatment regimens may vary as well, and depend on the stage of severe sepsis and its consequences. The clinician will be best suited to make decisions on the best regimen to use based on the positive determination of the existing severe sepsis, the use of antibiotics and the known efficacy and toxicity (if any) of the therapeutic formulations.

By using lactoferrin, in particular, an improvement of severe sepsis is obtainable.

The improvement is any observable or measurable improvement. Thus, one of skill in the art realizes that a treatment may improve the patient or subject's condition, but may not be a complete cure of the disease. In certain aspects, the composition is administered in an effective amount to decrease, reduce, inhibit or abrogate levels of severe sepsis.

A further embodiment of the present invention is a method of treating severe sepsis comprising the step of supplementing a mucosal immune system by increasing the amount of lactoferrin in the gastrointestinal tract. Preferably, the lactoferrin is administered orally.

Still yet, a further embodiment is a method of enhancing a mucosal immune response in the gastrointestinal tract in a subject comprising the step of administering orally to said subject a lactoferrin composition. The composition contains lactoferrin alone or in combination with a metal chelator, such as EDTA. It is envisioned that lactoferrin stimulates interleukin-18 in the gastrointestinal tract, which enhances immune cells. It is known by those of skill in the art that IL-18 is a Th₁ cytokine that acts in synergy with interleukin-12 and interleukin-2 in the stimulation of lymphocyte IFN-gamma production. Production of other cytokines may also be altered for example, but not limited to IL-1, IL-2, IL-10, IL-12 or IFN-gamma. It is also envisioned that lactoferrin stimulates interleukin-18 following oral administration, which inhibits pro-inflammatory cytokines, i.e., IL-4, IL-5, IL-6, IL-8 and TNF-alpha.

Yet further, it is envisioned that oral administration of lactoferrin in combination with a metal chelator, such as EDTA, enhances the amount of metal ion that is sequestered and therefore enhances the effectiveness of lactoferrin in enhancing the immune system.

### C. COMBINATION TREATMENTS

In order to increase the effectiveness of the composition, it may be desirable to combine these compositions and methods of the invention with a known agent effective in the treatment or prevention of bacteremia, sepsis, septic shock and related conditions, for example known agents to treat bacterial infections, e.g., antibiotics and agents to treat inflammation. In some embodiments, it is contemplated that a conventional therapy or agent, including but not limited to, a pharmacological therapeutic agent may be combined with the composition of the present invention.

The composition of the present invention may precede, be co-current with and/or follow the other agent(s) by intervals ranging from minutes to weeks. In embodiments where the composition of the present invention, and other agent(s) are applied separately to a cell, tissue or organism, one would generally ensure that a significant period of time did not expire between the time of each delivery, such that the composition and agent(s) would still be able to exert an advantageously combined effect on the cell, tissue or organism.

Various combination regimens of the composition and one or more agents are employed. One of skill in the art is aware that the composition of the present invention and agents can be administered in any order or combination. In other aspects, one or more agents may be administered substantially simultaneously, or within about minutes to hours to days to weeks and any range derivable therein, prior to and/or after administering the composition.

Administration of the composition to a cell, tissue or organism may follow general protocols for the administration of cardiovascular therapeutics, taking into account the toxicity, if any. It is expected that the treatment cycles would be repeated as necessary. In particular embodiments, it is contemplated that various additional agents may be applied in any combination with the present invention.

Pharmacological therapeutic agents and methods of administration, dosages, etc. are well known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antimicrobial agent, an anti-inflammatory agent, an antithrombotic/fibrinolytic agent, a blood coagulant, an antiarrhythmic agent, an antihypertensive agent, a vasopressor, or agents to treat metabolic acidosis. In certain aspects of the present invention, antimicrobial agents, e.g., antibiotics are used in combination with the composition of the present invention. Examples of specific antibiotics that can be used include, but are not limited to, erythromycin, nafcillin, cefazolin, imipenem, aztreonam, gentamicin, sulfamethoxazole, vancomycin, ciprofloxacin, trimethoprim, rifampin, metronidazole, clindamycin, teicoplanin, mupirocin, azithromycin, clarithromycin, ofloxacin, lomefloxacin, norfloxacin, nalidixic acid, sparfloxacin, pefloxacin, amifloxacin, gatifloxacin, moxifloxacin, gemifloxacin, enoxacin, fleroxacin, minocycline, linezolid, temafloxacin, tosufloxacin, clinafloxacin, sulbactam, clavulanic acid, amphotericin B, fluconazole, itraconazole, ketoconazole, and nystatin. Other examples of antibiotics, such as those listed in Sakamoto et al, U.S. Pat. No. 4,642,104 herein incorporated by reference will readily suggest themselves to those of ordinary skill in the art. Anti-inflammatory agents include, but are not limited to non-sterodial anti-inflammatory agents (e.g., naproxen, ibuprofen, celeoxib) and sterodial anti-inflammatory agents (e.g., glucocorticoids).

The invention of the present application may be partially characterized by reference to this nonexclusive list of exemplary items:
1. A method of treating a human subject suffering severe sepsis comprising administering to the human subject a human lactoferrin in a therapeutically effective amount to reduce the risk of death of the human subject from the severe sepsis.
2. The method of item 1, wherein the human subject has a baseline APACHE II score of ≦25 such as less than 21.
3. The method of items 1-2, wherein the severe sepsis comprises no more than one organ dysfunction.
4. The method of items 1-3, wherein the human subject has unimpaired cardiovascular functions.
5. The method of items 1-4 wherein the human subject is less than 18 years old, such as less than 15 years old, less than 12 years old, less than 3 years old or less than 60 days old.
6. The method of items 1-5 wherein the human lactoferrin is administered enterally in 1.5 milligrams to 100 gram doses every 8 hours, such as 1.5 grams per 8 hrs, optionally co-administered with an antacid compound.
7. The method of items 1-6 wherein the administration of the human lactoferrin is only by enteral routes of administration for gastrointestinal delivery of the human lactoferrin.

### D. FIGURES

FIG. 1 shows a summary of populations for efficiency analysis and safety analysis.

FIG. 2 shows an Apache II Score vs. 28-Day All-Cause Mortality.

FIG. 2A shows patients with no cardiovascular dysfunction.

FIG. 2B shows patients having cardiovascular dysfunction.

FIG. 3 shows a Kaplan-Meier analysis of 28-Day All-Cause Mortality (probability of survival vs. survival (days)).

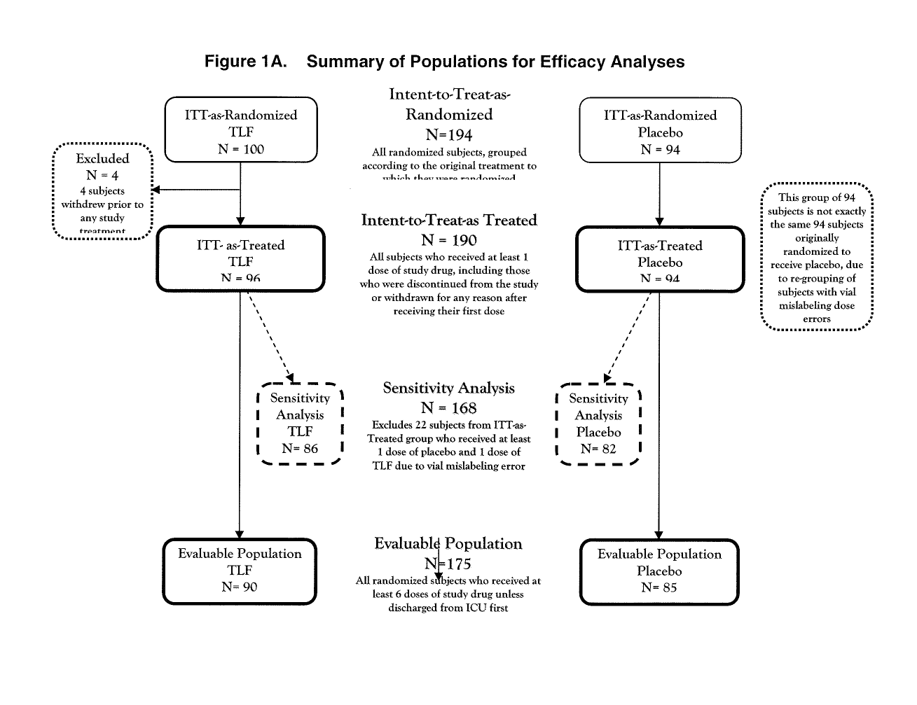

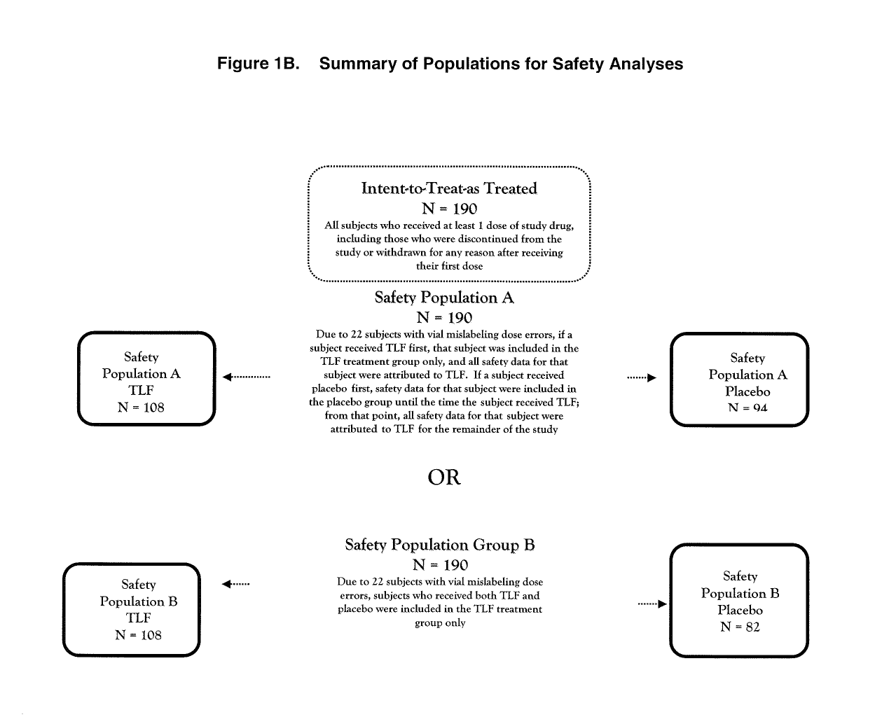

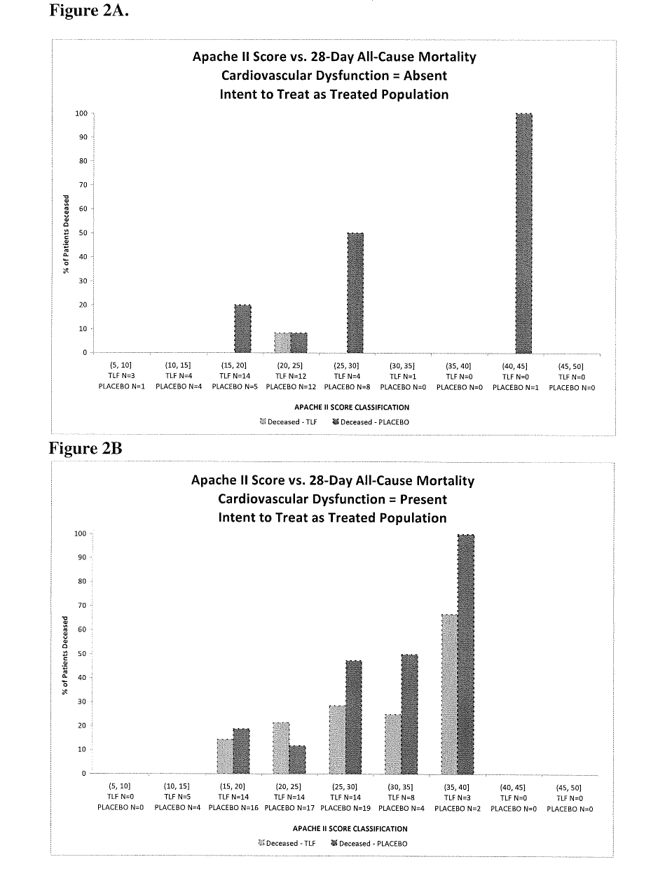

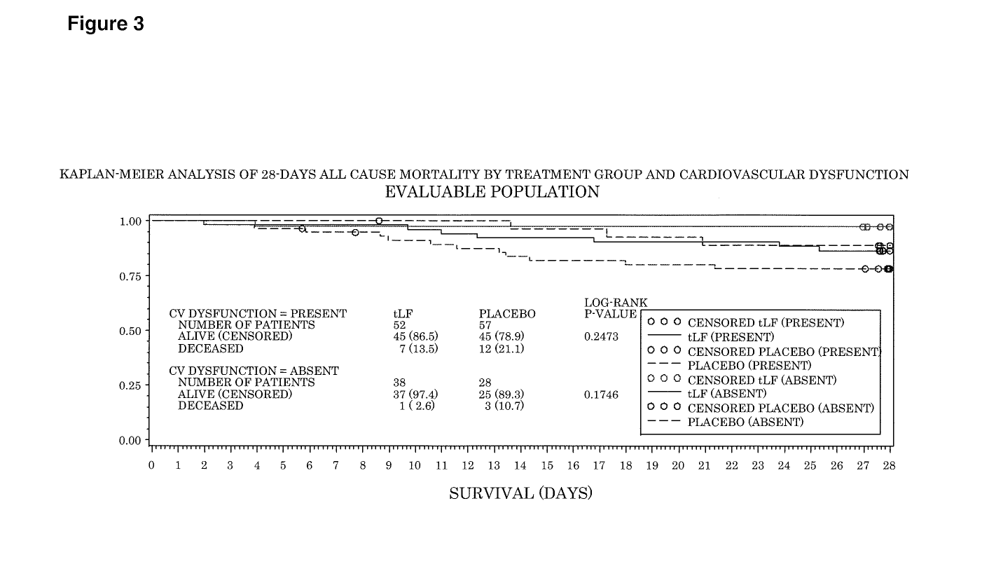

### E. EXAMPLES

The following examples are included to demonstrate preferred embodiments of the invention.

### Example 1

### A Phase 2 Randomized, Double-Blind, Placebo-Controlled Study of the Safety and Efficacy Of Talactoferrin Alfa in Patients with Severe Sepsis

Sepsis has been defined as infection, or suspected infection, accompanied by the presence of systemic inflammation. Severe sepsis has been defined as the presence of sepsis and 1 or more organ dysfunctions. Organ dysfunctions may include acute lung injury, coagulation abnormalities, thrombocytopenia, altered mental status, renal, liver or cardiac failure, or hypoperfusion with lactic acidosis. Organ dysfunction may be assessed by the Sepsis-related Organ Failure Assessment (SOFA) score.

Based on the animal studies in sepsis, and based on extensive safety experience, the dose chosen for this trial is 1.5 g lactoferrin every 8 hours.

A dose of 1.5 g appears to be effective, and higher doses do not appear to provide any additional benefit.

In preclinical studies in sepsis, we tested different administration schedules of talactoferrin. Talactoferrin administered three times a day appears to be more effective than talactoferrin administered two times a day. Hence, a dose and schedule of 1.5 g every 8 hours was used in this study.

Talactoferrin was provided to the patient as a 100 mg/mL solution in a phosphate buffer (pH 7), in individual 15-mL unit doses. Each 15-mL dose was administered orally or via other enteral route, 3 times a day (i.e., every 8 hours).

The talactoferrin dose level of 1.5 g administered every 8 hours (total daily dose of 4.5 g) was well-tolerated.

The patients were further classified with regard to presence or absence of cardiovascular dysfunction.

TABLE 1
Study Procedures Flow Chart

| | | | | | | | | | | Final Safety | | |
|---|---|---|---|---|---|---|---|---|---|---|---|---|
| | | | | | | | | | 672 hr. | Evaluation² | Follow- | Follow- |
| | Baseline | Active Treatment Period | | | | | | Study | (+48) | 28 (±2) | up | up |
| Study Days | −1 to 1 | 1-6 | 7 | 8-13 | 14 (±2) | 21 (±2) | 28 (+2) | Drug | Follow- | from Study | 3 mo. | 6 mo. |
| Study Weeks | | | 1 | | 2 | 3 | 4 | D/C¹ | up | Drug D/C | 12 | 24 |
| Onset of Severe Sepsis³ | X | | | | | | | | | | | |
| Informed Consent | X | | | | | | | | | | | |
| Demographics | X | | | | | | | | | | | |
| Inclusion/Exclusion | X | | | | | | | | | | | |
| APACHE II Evaluation | X | | | | | | | | | | | |
| Medical History | X | | | | | | | | | | | |
| Medication History | X | | | | | | | | | | | |
| Baseline Signs & | X | | | | | | | | | | | |
| Symptoms | | | | | | | | | | | | |
| Pregnancy Test | X | | | | | | | | | | | |
| Physical Examination | X | | | | | | X | X | | | | |
| Randomization⁴ | X | | | | | | | | | | | |
| Vital Signs | X | Daily | X | Daily | X | X | X | X | | | | |
| Weight (if easily obtained) | X | Daily | X | Daily | X | X | X | X | | | | |
| Hematology | X | Daily | X | Daily | X | X | X | X | | | | |
| AST, ALT, Alkaline | X | Day 1, 2, 3 | X | Day 10 | X | X | X | X | | | | |
| Phosphatase, | | | | | | | | | | | | |
| Total Bilirubin | | | | | | | | | | | | |
| INR, Albumin⁵ | X | | | | | | | | | | | |
| Serum Chemistry | X | Daily | X | Daily | X | X | X | X | | | | |
| Serum Lactate⁶ | X | Daily | X | | | | | | | | | |
| Input/Output Total Fluids and | X | Daily | X | Daily | X | X | | | | | | |
| Total Urine Output | | | | | | | | | | | | |
| Urine, Sputum, Endo. | X⁷ | | | | | | | | | | | |
| Aspirate, CSF, or | | | | | | | | | | | | |
| Stool Culture | | | | | | | | | | | | |
| Blood Culture | X⁸ | | | | | | | | | | | |
| Organ Function (SOFA) | X | Day 1, 2, 3 | X | Day 10 | X | X | X | X | | | | |
| Adverse Events | | Continuous | | | | | | X | | X | | |
| Concomitant & Antibiotic | | Continuous | | | | | | X | | X | | |
| Medications | | | | | | | | | | | | |
| Study Drug Administration⁹ | | Continuous | | | | | | | | | | |
| Cytokines, C-Reactive | X | Day 3 | X | Day 10 | X | X | X | X | | | | |
| Protein, and Procalcitonin | | | | | | | | | | | | |
| Survival Follow-up | | | | | | | | | X¹⁰ | X | X | X |

¹Visit occurs on day of last dose or upon ICU discharge, whichever is earlier. Required assessments performed ≦72 hours prior to this visit do not need to be repeated. Study Drug Discontinuation Vsit is not required if Study Day 28 Visit is completed.
²Visit occurs 28 days after the last dose of Study Drug. May be performed by telephone contact.
³Onset of severe sepsis must be within 24 hours prior to randomization as defined in Section 6.1
⁴Randomization/enrollment only after patient eligibility is verified through a centralized review and the patient qualifies for the study.
⁵Record at baseline, repeat as clinically indicated.
⁶Record at baseline and then daily until normalized, or Day 7 whichever is earlier. Repeat as clinically indicated.
⁷Perform only if clinically indicated. At 48-72 hours post randomization an assessment of the adequacy of initial empiric antibiotic therapy will be made for all baseline microbiological isolates
⁸Perform at baseline, repeat as clinically indicated. At 48-72 hours post randomization an assessment of the adequacy of initial empiric antibiotic therapy will be made for all baseline microbiological isolates
⁹First dose of Study Drug must be given ≦4 hours post-randomization. Doses should be given every 8 (±2) hours for 28 days or until discharge from the ICU, whichever is earlier
¹⁰Survival status must be documented at 672 (+48) hours post-first dose of Study Drug. If the Study Drug Discontinuation Visit occurs prior to Day 28, this may be performed via telephone contact

During the Active Treatment Period, patients received lactoferrin every 8 (±2) hours for 28 days or until discharge from the ICU, whichever is earlier. Lactoferrin was administered orally, or if the patient was not able to consume it orally, it was be administered enterally via a feeding tube (pre- or post-pyloric).

### Primary Analysis

The primary efficacy endpoint was analyzed based on comparison between the talactoferrin-treated group and the placebo group. Analysis of 28-day (672-hour post first dose of Study Drug) all-cause mortality was conducted for the ITT population using that Cochran-Mantel-Haenszel (CMH) test stratified by study center and the presence or absence of cardiovascular dysfunction.

### Secondary Analyses

As a secondary analysis, the primary endpoint of 28-day (672-hour post first dose of Study Drug) all-cause mortality was also analyzed for the Evaluable population. This endpoint was also compared across treatment groups using the Kaplan-Meier and Log-Rank test.
All secondary analyses were conducted on the ITT and Evaluable populations. The mean number of ICU, shock-free, ventilator-free, dialysis-free, organ-dysfunction-free, and vasopressor medication days were compared across treatment groups using two-sample t-tests. The incidence of additional organ failure/dysfunction, new infection and ICU discharge was compared across treatment groups using Fisher's exact test. Time-to-event outcomes were analyzed with Kaplan-Meier methods.

### Analysis of Safety

Adverse events, clinical laboratory data, and vital signs were analyzed to evaluate the safety of talactoferrin for all patients in the Safety-population from the first dose of Study Drug (active or placebo) through 4 weeks after the last dose of Study Drug.

### Introduction

This study was a double-blind, placebo-controlled, multicenter, 2-arm, Phase 2 clinical study of the efficacy and safety of enterally administered talactoferrin alfa (TLF) in patients with severe sepsis. 194 subjects were randomized to receive either TLF or placebo. Study treatments were administered orally (or through other enteral procedures) for a maximum of 28 days, or until the subject was discharged from the ICU, whichever occurred first. Safety was monitored daily while in the ICU and a final safety evaluation was made 4 weeks after the last dose of study drug. Subjects were contacted at 3 and 6 months post-randomization to determine survival status.

study OBJECTIVES and endpoints

### Primary Objective (Primary Efficacy Endpoint)

The primary objective was to determine 28-day (672-hour post first dose of study drug) all-cause mortality.

### Secondary Objectives (Secondary Efficacy Endpoint)

The secondary objectives were to determine the:
Number of ICU days for survivors
Proportion of shock-free days in the ICU
Proportion of ventilator-free days in the ICU
Proportion of dialysis-free days in the ICU
Proportion of organ dysfunction-free days in the ICU
Duration of use of vasopressor medications in the ICU
Incidence and severity of additional sepsis-related organ dysfunction in the ICU
Incidence of new infection in the ICU
Incidence of ICU discharge
3-month all-cause mortality
6-month all-cause mortality
Time to death
Safety and toxicity of enteral TLF in patients with severe sepsis

### Safety Endpoints

Number of treatment-emergent and study agent-related adverse events (AEs)
Number of serious adverse events (SAES)
Number of treatment discontinuations due to AEs
Number of AEs
Number of Grade 3-4 AEs

### Investigational Plan

### Overall Study Design and Plan Description

This was a double-blind, placebo-controlled study of enteral TLF in patients with severe sepsis. A total of 401 patients were screened to determine their eligibility for the study.
A total of 194 subjects met the eligibility criteria and were enrolled in the study. Subjects were randomized at a ratio of 1:1 to receive either TLF or placebo for up to 28 days or until discharge from the ICU, whichever occurred first. Of the 194 subjects enrolled: 100 were randomized to TLF and 94 were randomized to placebo. TLF and placebo arms were stratified by study center and by the presence or absence of cardiovascular dysfunction at the time of randomization.
The study enrolled subjects who had a diagnosis of sepsis and at least 1 organ dysfunction. Subjects received standard care, including any therapies which were appropriate, including, at the discretion of the primary physician, Xigris®. If the subject was not able to consume study drug orally, it was administered via other enteral procedures. After randomization, the patient was given the first dose of study drug as soon as possible (within 4 hours of randomization). Dosing was continued for a maximum of 28 days or until the subject was discharged from the ICU, whichever occurred first.
Safety was monitored daily in the ICU and a final safety evaluation was made 4 weeks after the last dose of study drug. Subjects were contacted at 3 months and 6 months post-randomization to determine survival status.

### Selection of Study Population

### Inclusion Criteria

Subject candidates were required to fulfill all of the following inclusion criteria to be eligible for participation in the study:
1. Age≧18 years
2. Onset of severe sepsis within the previous 24 hours as defined by meeting all of the following criteria (A, B, and C):
   A. Known or suspected infection. Suspected infection was based on evidence of infection such as the presence of 1 or more of the following: a) white blood cells (WBCs) in a normally sterile body fluid; b) perforated viscus; c) radiographic evidence of pneumonia; or d) a syndrome associated with a high likelihood of infection (eg, ascending cholangitis).
   B. The presence of at least 2 of the following criteria for the manifestation of systemic inflammation, one of which must be changes in body temperature or WBC (including immature neutrophils [“bands”]):
      Body temperature: i) hyperthermia as indicated by a temperature of ≧38° C. (100.4° F.); or ii) hypothermia as indicated by a core temperature ≦36° C. (96.8° F.). Hypothermia was defined by a temperature obtained via rectal or central catheter measurement, and not by oral, tympanic or axillary measurement
      Heart rate ≧90/min, except in patients with a medical condition known to increase the heart rate or those receiving treatment that would prevent tachycardia
      Respiratory rate ≧20/min or PaCO₂ ≦32 mmHg; or the use of mechanical ventilation for an acute respiratory process
      WBC ≧12,000/mm³ or ≦4,000/mm³, or >10% immature neutrophils “bands”,
      and
   C. At least 1 acute organ dysfunction due to sepsis, that was newly developed and not explained by other disease processes or the effects of treatment, defined as follows:
      Cardiovascular—the requirement for vasopressors (vasopressors were defined as dopamine ≧5 μg/kg/min or any dose of norepinephrine, epinephrine, phenylephrine, or vasopressin, with the intent to support blood pressure. Dobutamine and dopexamine were not considered vasopressors.), in addition to adequate fluid resuscitation (adequate fluid resuscitation was defined as one of the following: i) a minimum of a 20 mL/kg (ideal body weight) intravenous fluid challenge (crystalloid or equivalent colloid); or ii) if measured, a central venous pressure (CVP) ≧8 mm Hg; or iii) a pulmonary artery occlusion pressure (PAOP) ≧12 mm Hg.), to maintain a MAP >65 mmHg or a systolic pressure >90 mmHg
      Respiratory—PaO₂/FiO₂ ratio ≦250, or ≦200 if the lung was the primary site of infection, and, if measured, a capillary wedge pressure not suggestive of central venous volume overload
      Renal—i) an absolute increase in serum creatinine of ≧0.3 mg/dL from baseline, or ii) a ≧50% increase in serum creatinine from baseline, or iii) a urine output <0.5 mL/kg/hr for ≧1 hour. The preceding criteria must have been met despite adequate fluid resuscitation (defined as above for cardiovascular dysfunction).
      Hematologic—platelet count <80,000/mm³ or decreased by ≧50% from the highest value recorded within 3 days prior to randomization
      Metabolic—serum lactate >4.0 mm/L (or equivalent in other units)

### Treatments

### Treatments Administered

The investigational drug TLF is a recombinant form of the protein human lactoferrin. It is produced by Aspergillus niger var. awamori under cGMP. TLF is structurally and functionally equivalent to the native lactoferrin protein from human milk, as demonstrated by a comparison of the 3-dimensional structure, molecular weight, biological activity, and other physicochemical properties, differing only in the nature of glycosylation.
TLF was supplied as a solution in a phosphate-based buffer at a concentration of 100 mg/mL in vials for oral administration. Each subject received a dose of 1.5 g (1 vial, 15 mL) TLF every 8 (±2) hours for a total daily dose of 4.5 g.
The placebo contained the same phosphate-based buffer used for TLF, plus FD&C/EU-grade dyes suitable for oral use to mimic the color of the vialed drug product. The volume of placebo solution, administered every 8 (±2) hours, was 15 mL

### Treatment Assignment

Subjects were randomized at an approximately 1:1 ratio to receive either TLF or placebo as follows:
   100 subjects—1.5 g (1 vial TLF) every 8 (±2) hours
   94 subjects—1.5 g (1 vial placebo) every 8 (±2) hours
Four subjects were randomized but did not receive the first dose of study drug, and were withdrawn
The ITT (intent-to-treat)-as-Treated population consisted of a total of 190 subjects, 96 who received TLF and 94 who received placebo during the first week of treatment.

### Selection of Doses in the Study

A dose of 4.5 g/day of TLF was chosen for this study, based on preclinical data and prior clinical experience with TLF in which cancer patients were administered doses of 1.5 g/day to 9 g/day (approximately 21 mg/kg/day to 129 mg/kg/day) with no apparent dose-response relationship.

The 4.5 g daily oral dose was well-tolerated.

### Selection of Timing of Dose for Each Subject

Oral TLF has been administered to over 600 subjects in 24 clinical trials conducted in the US and around the world. These studies evaluated various dosing schedules with between 1 and 5 doses per day. Based on these results, the dosing schedule selected for this study was 3 doses per day, with at least 30 minutes before or 60 minutes after ingestion of any meals.

### Blinding

This was a double-blind study.

### Prestudy Evaluations and Baseline (Study Day −1 to 1)

Documentation of the onset of severe sepsis within the 24 hours prior to randomization

The following assessments may be performed any time within the 24 hours prior to randomization:
Review of inclusion/exclusion criteria for study
Acute Physiology and Chronic Health Evaluation (APACHE) II for the 24 hours prior to randomization
   672 hours (+48 hours) Post First Dose Study Drug Survival

Survival was determined for all subjects at 672 hours (+48 hours) post the first dose of study drug.

Final Safety Evaluation (28 days post last dose of study drug)
For the Final Safety Visit, the following information was obtained via telephone contact:
   Survival status
   Recorded any changes in concomitant medications
   Recorded any AEs which had occurred during the 28 days since last dose of study drug.

### Efficacy and Safety Measurements Assessed

Evaluation of Severe Sepsis
Parameters of severe sepsis were determined.

Evaluation of Organ Function
This was determined using the inclusion criteria ‘and the Sepsis-related Organ Failure Assessment (SOFA) Score while on study.

Adverse Events
An AE was defined as any untoward medical occurrence in a patient after initiation of study drug treatment without regard to the possibility of a causal relationship with the study drug. An AE could therefore have been any unfavorable and unintended sign, symptom, or disease temporally associated with the use of the investigational product, whether or not it was considered related to the investigational product. This included any side effects, injury, toxicity, or sensitivity reaction, and may have included a single symptom or sign, a set of related symptoms or signs, or a disease. An AE could also have been any laboratory abnormality judged to be clinically significant by the Investigator or Sub-investigator(s) which had worsened when compared to Baseline.
A Serious Adverse Event (SAE) was any AE that resulted in any of the following:
   death,
   life-threatening event,
   hospitalization or prolongation of hospitalization,
   a persistent or significant disability/incapacity,
   congenital anomaly/birth defect, or
i.an event that required intervention to prevent any one of the other outcomes listed above (based on medical judgment)

### Appropriateness of Measures

Approximately 190 volunteers were to be enrolled in order to achieve a sufficient evaluable population. In order to detect a decrease in mortality (from 30% to 17% mortality, a ≧43% relative decrease), a sample size of 95 patients per arm provided 80% power with a 1-sided p value of <0.1.

All-cause mortality at 28 days post-treatment, the primary endpoint, is commonly used in clinical trials evaluating treatments for severe sepsis. The secondary endpoints evaluated in this study were considered to be acceptable secondary endpoint measures for a phase 2 study. Assessments of mortality at the 3-month and 6-month timepoints, exploratory secondary efficacy endpoints, represent reasonable time intervals for longer-term follow-up of the patients. Measurements associated with the remaining secondary endpoints were selected to explore the possibility of effects of TLF on selected symptoms of sepsis or other response-related variables. These include measures of the proportion of shock-free, ventilator-free, and dialysis-free days in the ICU, and the incidence of organ failure/dysfunction and new infection in the ICU.

### Statistical and Analytical Plans

Standard statistical methods were employed to analyze all data. The following techniques were used: descriptive statistics, two-sample t-test, paired t-test, Fisher's exact test, Kaplan-Meier, Cox proportional hazards, Log-Rank test, logistic regression, odds ratio, Cochran-Mantel-Haenszel (CMH), Breslow Day test, and two-proportion binomial test.

All tests of safety and efficacy endpoints were declared statistically significant if the calculated p-value was less than or equal to 0.05, and appear as two-sided p-values. P-values between 0.05 and 0.10 were declared borderline statistically significant.

Summary statistics consist of numbers and percentages of responses in each category for discrete measures, and of means, medians, standard deviations, 90% confidence intervals, minimum, and maximum values for continuous measures.

Version 9.1 of the SAS® statistical software package was used to provide all statistical analyses.

### Determination of Sample Size

A population of patients with severe sepsis typically demonstrates at least 30% incidence of 28-day mortality. A sample size of 95 patients per treatment arm was chosen to provide 80% power to detect a ≧43% relative decrease in mortality (from 30% to 17% mortality) with a 1-sided P-value of <0.1. The proportion of patients with a genitourinary site of known or suspected infection was capped at 15%.

### Subject Disposition

Of the 194 randomized subjects, 4 were withdrawn prior to receiving study treatment. A total of 190 subjects received at least 1 dose of study medication.

All study medication was assigned to subjects by cartons, each of which contained sufficient medication to provide for one week of treatment (3 doses per day for 7 days). Those patients who received both TLF and placebo were on study longer than 7 days. A summary of Subject Disposition overall and by treatment group is presented in Table 2 below.

TABLE 2
Subject Disposition By Treatment Group And Overall

| | Treatment Group | | | | |
|---|---|---|---|---|---|
| | TLF | | Placebo | | Overall |
| Subject Disposition | N | (%) | N | (%) | N |
| Subjects screened | | | | | 401 |
| Subjects in the ITT-as- | 100 | (51.5) | 94 | (48.5) | 194 |
| Randomized^{a} | | | | | |
| Subjects in the ITT-as-Treated^{b} | 96 | (50.5) | 94 | (49.5) | 190 |
| Subjects in the Evaluable | 90 | (51.4) | 85 | (48.6) | 175 |
| Population^{c} | | | | | |
| Subjects in the Sensitivity | 86 | (51.2) | 82 | (48.8) | 168 |
| Analysis for the ITT-as- | | | | | |
| Treated^{d} | | | | | |
| Subjects in Safety Population A^{e} | 108 | (56.8) | 94 | (49.5) | 190 |
| Subjects in Safety Population B^{f} | 108 | (56.8) | 82 | (43.2) | 190 |

Reference: Table 14.1.1
^{a}All randomized subjects are included in the treatment group to which they were randomized.
^{b}All randomized subjects who received at least 1 dose of study drug. Subjects are included in the treatment group of the study drug received during the first week of treatment.
^{c}All randomized subjects who received a minimum of 6 doses of study drug, unless discharged earlier from the ICU. Treatment group assignment is that described for ITT-as-Treated.
^{d}All ITT-as-treated subjects with the exception of subjects who received both TLF and placebo.
^{e}All ITT-as-treated subjects. Treatment group assignment is that described for ITT-as-treated, unless subject received both TLF and placebo. If subject received TLF first, that subject is included in the TLF treatment group only. If subject received placebo first, that subject is included in the placebo group until the time that patient received TLF; from that point onward, the subject is included in the TLF treatment group.
^{f}All ITT-as-treated subjects. Treatment group assignment is that described for ITT-as-treated, unless subject received both TLF and placebo; if so, then that subject is included in the TLF treatment group only.

Twenty-two (22.9%) subjects treated with TLF and 36 (38.3%) subjects treated with placebo were discontinued from the study. Of the subjects treated with TLF, 9 (40.9%) were discontinued during the active treatment period and 13 (59.1%) during the survival follow-up. A similar situation occurred in the placebo-treated group: 14 (38.9%) were discontinued during the active treatment period and 22 (61.1%) during the survival follow-up.

The most common reason for premature discontinuation in both treatment groups was death, occurring in 16 (72.7%) of the 22 discontinued subjects treated with TLF and 27 (75.0%) of the 36 discontinued subjects treated with placebo.

TABLE 3
Reasons for Study Discontinuation (ITT-as-Treated Population)

| | Treatment Group | | | |
|---|---|---|---|---|
| | TLF | | Placebo | |
| | N = 96 | | N = 94 | |
| Subject Disposition | n | (%) | n | (%) |
| Subjects who have completed survival | 74 | (77.1) | 58 | (61.7) |
| follow-up period (6 months post last | | | | |
| dose of study drug) | | | | |
| Subjects discontinued from the study | 22 | (22.9) | 36 | (38.3) |
| Reasons for discontinuation | | | | |
| During active treatment period | 9 | (40.9) | 14 | (38.9) |
| Died^{a} | 7 | (77.8) | 12 | (85.7) |
| Withdrew consent during active treatment | 2 | (22.2) | 2 | (14.3) |
| period | | | | |
| During survival follow-up period | 13 | (59.1) | 22 | (61.1) |
| ≦28 days post-treatment | 5 | (38.5) | 14 | (63.6) |
| Withdrew consent | 0 | | 2 | |
| Died | 3 | | 9 | |
| Lost to follow-up | 0 | | 2 | |
| Other | 2 | | 2 | |
| >28 days post-treatment | 8 | (61.5) | 8 | (36.4) |
| Withdrew consent | 2 | | 2 | |
| Died | 6 | | 6 | |
| Lost to follow-up | 0 | | 0 | |
| Other | 0 | | 0 | |

Reference: Table 14.1.4.3
^{a}Three subjects who withdrew prior to the 672-hour survival endpoint were imputed as deceased due to having organ dysfunction at the time of last contact.
^{b}Reasons reported as “Died after readmission to ICU (Study Drug was stopped previously after discharged from ICU)” and “Died after life support withdrawn.”
^{c}Reasons reported as “Family decision to withdraw all treatment” and “MD decision to withdraw patient from study.”

### Efficacy Evaluation

### Data Sets Analyzed

Of the 194 subjects randomized, 190 were treated with study medication and comprise the ITT-as-Treated population. Safety and efficacy analyses were performed on data from all 190 subjects.

### Efficacy Populations

### Intent-to-Treat (ITT) as Randomized Population

The ITT-as-Randomized population included all randomized patients. This population consists of 194 subjects (TLF N=100, placebo N=94).

### Intent-to-Treat (ITT) as Treated Population

The ITT-as-Treated population included all randomized patients who received at least 1 dose of study drug, including those who were discontinued from the study or withdrawn for any reason after receiving their first dose. This population is comprised of 190 subjects, and excludes 4 subjects from the ITT-as-Randomized population who were withdrawn prior to receiving any study medication (TLF N=96, placebo N=94).

### Demographic and Other Baseline Characteristics

### Demographic Characteristics

For the ITT-as-Treated population, no statistically significant differences were noted between the treatment groups with regard to age, gender, race, ethnicity, height, or weight. The mean (±SD) age was 58.1±17.4 years for the TLF-treated group, and 60.9±15.9 years for the placebo-treated group. The ratio of males to females was approximately 1:1 in each treatment group. Nearly 75% of the subjects in each group were white, and approximately 15% were Black/African American.

### Baseline Characteristics

Baseline characteristics deemed relevant to the study were compared between the two treatment groups in the ITT-as-Treated population. Evaluations at baseline are listed in the Study Procedure Flow Chart (Table 1). In addition to standard evaluations such as physical examinations, vital signs, weight, and height; hematology and serum chemistry, and liver function tests (LFTs), baseline evaluations included determination of organ dysfunctions and use of concomitant medications; cultures of urine, sputum or endotracheal aspirate, blood, cerebrospinal fluid (CSF), and stool; and determination of SOFA scores, APACHE II scores, and Charlson Co-morbidity Scores.

The baseline characteristics appear similar between the 2 treatment groups. In addition to these measurements, a comparison of organ dysfunction demonstrates no statistically significant differences between treatment groups at baseline. The mean number of organs with dysfunction was 1.9±1.0 (SD) in the TLF-treated group, and 2.1±1.1 (SD) in the placebo-treated group.

Because of the nature of severe sepsis, characteristics related to bacterial infection were compared between treatment groups. No significant differences were noted. In the ITT-as-Treated population, the lung was the most frequent site of infection: 44 subjects (45.8%) in the TLF group and 49 subjects (52.1%) in the placebo group. This correlates with the high incidence of respiratory active medical histories reported (see Section 11.2.2). The second most-commonly reported site was blood: 37 subjects (38.5%) in the TLF group and 26 subjects (27.7%) in the placebo group.

Negative culture results were noted at baseline in 51 subjects (53.1%) receiving TLF and in 45 subjects (47.9%) receiving placebo. Subjects treated with TLF had a lower incidence of gram stain positive results (33 subjects, 73.3%) than the placebo groups (41 subjects, 83.7%). The gram stain positive organisms commonly responsible for infection and sepsis, Methicillin-resistant Staphylococcus aureus and Streptococcus pneumonia, were detected at similar frequencies in both populations. The incidence of “Other” organisms was approximately double in placebo-treated subjects compared to TLF-treated subjects in both populations. Results for the Evaluable population were similar to the ITT-as-Treated population in all categories of infection-related baseline characteristics.

TABLE 4
Baseline Characteristics by Treatment Group (ITT-as-
Treated Population)

| | Treatment Group | |
|---|---|---|
| | TLF | Placebo |
| Baseline Characteristic | N = 96 | N = 94 |
| Use of Xigris - n (%) | | |
| Yes | 9 (9.4) | 5 (5.3) |
| No | 87 (90.6) | 89 (94.7) |
| Use of Xigris on or before first | | |
| treatment - n (%) | | |
| Yes | 6 (6.3) | 4 (4.3) |
| No | 90 (93.8) | 90 (95.7) |
| Use of Corticosteroids - n (%) | | |
| Yes | 55 (57.3) | 56 (59.6) |
| No | 41 (42.7) | 38 (40.4) |
| Use of Corticosteroids on or before | | |
| first treatment - n (%) | | |
| Yes | 37 (38.5) | 44 (46.8) |
| No | 59 (61.5) | 50 (53.2) |
| Use of Statin - n (%) | | |
| Yes | 40 (41.7) | 40 (42.6) |
| No | 56 (58.3) | 54 (57.4) |
| Use of Statin on or before first | | |
| treatment - n (%) | | |
| Yes | 33 (34.4) | 37 (39.4) |
| No | 63 (65.6) | 57 (60.6) |
| Use of Antimicrobial agent including | | |
| antifungals - n (%) | | |
| Yes | 96 (100.0) | 94 (100.0) |
| No | 0 (0.0) | 0 (0.0) |
| Use of Antimicrobial agent including | | |
| antifungals on or before first | | |
| treatment - n (%) | | |
| Yes | 95 (99.0) | 94 (100.0) |
| No | 1 (1.0) | 0 (0.0) |
| Antibiotics started within 1 hour of | | |
| meeting the criteria for severe | | |
| sepsis - n (%) | | |
| Yes | 88 (91.7) | 90 (95.7) |
| No | 8 (8.3) | 4 (4.3) |
| Use of Early Goal Directed Therapy - | | |
| n (%) | | |
| Yes | 65 (67.7) | 69 (73.4) |
| No | 31 (32.3) | 25 (26.6) |
| SOFA Scores - n (%) | | |
| Mean ± SD | 8.7 ± 3.9 | 9.0 ± 3.3 |
| Median (Min, Max) | 9.0 (0, 18) | 9.0 (3, 19) |
| APACHE Scores - n (%) | | |
| Mean ± SD | 22.5 ± 6.5 | 23.1 ± 5.9 |
| Median (Min, Max) | 22.5 (8, 37) | 23.0 (9, 41) |
| Charlson Co-Morbidity Scores - | | |
| n (%) | | |
| Mean ± SD | 2.7 ± 3.0 | 2.6 ± 2.8 |
| Median (Min, Max) | 2.0 (0, 18) | 2.0 (0, 13) |

Reference Tables 14.1.5.1, 14.1.7.1, 14.1.8.1, 14.1.16
^{a}

### Efficacy Results and Tabulation of Individual Subject Data

### Analysis of Efficacy

Prospectively defined analysis populations were identified for efficacy evaluations. Analysis of primary endpoints using logistic regression was conducted on all 4 Efficacy populations (ITT-as-Treated, Evaluable, ITT-as-Randomized, and Sensitivity Analysis populations); Kaplan-Meier Analysis was conducted on ITT-as-Treated and Evaluable populations. Secondary efficacy endpoints were analyzed for the ITT-as-Treated and Evaluable populations.

### Primary Efficacy Endpoint Analysis

### Primary Efficacy Endpoint

The primary efficacy endpoint, 28-day (672-hour post first dose of study drug) all-cause mortality (ACM), was analyzed by treatment group using logistic regression and Kaplan-Meier Analysis, and stratified by the presence or absence of cardiovascular dysfunction and by study site. Results stratified by study site could not be analyzed due to the relatively large number of study sites, which reduced the power of the study.

Prior to unblinding, the study team determined whether each subject lost to follow-up had sepsis-related organ dysfunction which required support at the time of last contact. Prior to Day 28, 3 subjects with organ dysfunction at the time of last contact were either lost to follow-up or withdrew consent. These subjects (one received TLF and 2 received placebo) were considered deceased for the purpose of analysis. One subject treated with placebo and lost to follow-up prior to day 28 was free of organ dysfunction at the time of last contact; the subject was counted as alive for analysis purposes.

### Primary Efficacy Endpoint Analyses Results

### Logistic Regression Analysis

In the ITT-as-Treated population, administration of TLF was associated with a 45% decrease in mortality at 28 days post treatment than that observed in patients receiving placebo (14 deaths, 14.6% deceased in the group receiving TLF; 25 deaths, 26.6% deceased in the group receiving placebo; p=0.0429, univariate logistic regression). In the absence of cardiovascular (CV) dysfunction, mortality at 28 days in the TLF-treated subjects was 88% lower than that in the placebo-treated group (1 death, 2.6% deceased for TLF vs. 7 deaths, 22.6% deceased for placebo). For subjects with cardiovascular dysfunction, an improvement in survival was detected: 13 (22.4%) TLF-treated subjects and 18 (28.6%) placebo-treated subjects were deceased at 28 days. When including CV dysfunction in the logistic regression analysis, the effect of TLF was borderline statistically significant, based on a 2-sided logistic regression (p-value of 0.0572).

Results for the Evaluable Population were similar: TLF-treated subjects demonstrated a statistically significant 56% decrease in mortality overall at 28 days when compared to placebo-treated subjects (2-sided logistic regression test; p-value=0.0404), from 20.0% mortality to 0%. When CV dysfunction was included in the logistic regression analysis, the effect of TLF was borderline statistically significant, based on a 2-sided logistic regression (p-value of 0.0575).

Results in the Sensitivity Analysis for the ITT-as-Treated population demonstrated a less significant effect of TLF on mortality: 13 deaths, 15.1% mortality for subjects receiving TLF, which is 41% lower than the 25.9% mortality (21 deaths) observed in the placebo-treated subjects overall at 28 days. These effects of TLF were borderline statistically significant, based on a 2-sided logistic regression test (p=0.0937).

The effects of TLF treatments on death rates were similar in the ITT-as-Randomized and ITT-as-Treated populations in each category.

TABLE 5
Comparison of All-Cause 28-Day Mortality Across Treatment Groups and
Stratified by Presence or Absence of Cardiovascular Dysfunction at Baseline
(ITT-as Treated, Evaluable, and Sensitivity Analysis Populations)

| | ITT-as-Treated Population | | | Evaluable Population | | | Sensitivity Analysis Population | | |
|---|---|---|---|---|---|---|---|---|---|
| | Treatment Group | | | Treatment Group | | | Treatment Group | | |
| | TLF | Placebo | | TLF | Placebo | | TLF | Placebo | |
| | N = 96 | N = 94 | | N = 90 | N = 85 | | N = 86 | N = 82 | |
| Primary Endpoint | n (%) | n (%) | P value | n (%) | n (%) | P value | n (%) | n (%) | P value |
| Overall Population^{c} | | | | | | | | | |
| Alive | 82 (85.4) | 69 (73.4) | | 82 (91.1) | 68 (80.0) | | 73 (84.9) | 60 (74.1) | |
| Deceased | 14 (14.6) | 25 (26.6) | 0.0429^{a} | 8 (8.9) | 17 (20.0) | 0.0404^{a} | 13 (15.1) | 21 (25.9) | 0.0937^{a} |
| Cardiovascular Dysfunction^{c} | | | | | | | | | |
| Present at Baseline | | | | | | | | | |
| n | 121 | | | 109 | | | 104 | | |
| Alive | 45 (77.6) | 45 (71.4) | | 45 (86.5) | 44 (77.2) | | 39 (76.5) | 37 (69.8) | |
| Deceased | 13 (22.4) | 18 (28.6) | 0.0572^{b} | 7 (13.5) | 13 (22.8) | 0.0575^{b} | 12 (23.5) | 16 (30.2) | 0.1126^{b} |
| Absent at Baseline | | | | | | | | | |
| n | 69 | | | 66 | | | 63 | | |
| Alive | 37 (97.4) | 24 (77.4) | | 37 (97.4) | 24 (85.7) | | 34 (97.1) | 23 (82.1) | |
| Deceased | 1 (2.6) | 7 (22.6) | | 1 (2.6) | 4 (14.3) | | 1 (2.9) | 5 (17.9 | |

Reference: Tables 14.2.1.1, 14.2.1.3, 14.2.1.4
^{a}P-values for treatment based on univariate logistic regression analysis
^{b}P values for treatment based on logistic regression analysis, including cardiovascular dysfunction in the model
^{c}Three subjects lost to follow-up or who withdrew consent prior to Day 28 having organ dysfunction at the time of last contact were treated as not having survived (1 TLF and 2 placebo). If the subject was free of organ dysfunction at the time of last contact, the subject was considered alive (0 TLF and 1 placebo)

Kaplan-Meier Analysis
The Kaplan-Meier analysis of 28 day ACM for the ITT-as-Treated population revealed a 45% decrease in mortality in the TLF-treated group compared to the placebo-treated group: 18 deaths, 18.8% deceased in the TLF-treated subjects and 29 deaths, 30.9% deceased in subjects treated with placebo. Results from analyses by cardiovascular dysfunction are similar to those from logistic regression analyses.
The number of subjects who died was insufficient to estimate median survival times for comparison of these treatment groups. However, the overall results are consistent with those of logistic regression analyses, in which administration of TLF improved survival at 28 days post treatment.
Similar results were obtained from Kaplan-Meier analysis of 28 day ACM for the Evaluable population, but with only borderline statistical significance (p=0.0888).
Results of the Cox Proportional Hazards Analysis of Time to Death also show similar trends (Table 10), demonstrating a statistically significant (p=0.0471) increase in survival times associated with TLF administration in the ITT-as-Treated population. When stratified by CV dysfunction, the effects of TLF on mortality are only borderline significant (p=0.0760). TLF has no significant effect on mortality in the Evaluable population.
Overall, administration of TLF is associated with lower 28-day all-cause mortality than that observed following treatment with placebo in the ITT-as-Treated population.

TABLE 6
Analyses of 28 Day Survival Time by Kaplan-Meier and Cox Proportional Hazards Methods (ITT-as-
Treated and Evaluable Populations)

| | ITT-as Treated Population | | | Evaluable Population | | |
|---|---|---|---|---|---|---|
| | Treatment Group | | | Treatment Group | | |
| | TLF | Placebo | P | TLF | Placebo | P |
| Kaplan-Meier Analysis of Time to Death | N = 96 | N = 94 | value^{a} | N = 90 | N = 85 | value^{a} |
| Overall | | | | | | |
| N | 96 | 94 | 0.0440 | 90 | 85 | 0.0888 |
| Alive (Censored) | 78 (81.3) | 65 (69.1) | | 77 (85.6) | 65 (76.5) | |
| Deceased | 18 (18.8) | 29 (30.9) | | 13 (14.4) | 20 (23.5) | |
| Cardiovascular Dysfunction | | | | | | |
| Present | | | | | | |
| N | 58 | 63 | 0.2782 | 52 | 57 | 0.2115 |
| Alive (Censored) | 43 (74.1) | 41 (65.1) | | 42 (80.8) | 41 (71.9) | |
| Number Deceased | 15 (25.9) | 22 (34.9) | | 10 (19.2) | 16 (28.1) | |
| Absent | | | | | | |
| N | 38 | 31 | 0.0688 | 38 | 28 | 0.3726 |
| Alive (Censored) | 35 (92.1) | 24 (77.4) | | 35 (92.1) | 24 (85.7) | |
| Deceased | 3 (7.9) | 7 (22.6) | | 3 (7.9) | 4 (14.3) | |

| | ITT-as Treated Population | | | Evaluable Population | | |
|---|---|---|---|---|---|---|
| Cox Proportional | | Hazards | | | Hazards | |
| Hazards Analysis of Time to Death | P-value^{b} | Ratio | 95% CI | P-value^{b} | Ratio | 95% CI |
| Treatment Stratified by CV Dysfunction | 0.0760 | 0.5861 | (0.325, 1.057) | 0.1335 | 0.5850 | (0.290, 1.178) |
| Treatment | 0.0471 | 0.5510 | (0.306, 0.993) | 0.0936 | 0.5501 | (0.274, 1.106) |

Reference Tables 14.2.14.1-4
^{a}Two-sided Log Rank Test P-value
^{b}Two-sided Wald Test P-value (Hazards Ratio is Placebo/Treatment)
^{c}Three subjects lost to follow-up or who withdrew consent prior to Day 28 having organ dysfunction at the time of last contact were treated as not having survived (1 TLF and 2 placebo). If the subject was free of organ dysfunction at the time of last contact, the subject was considered alive (0 TLF and 1 placebo)

11.4.1.2 Secondary Efficacy Endpoint Analysis

### Secondary Efficacy Endpoints

The secondary efficacy analyses were conducted on the ITT-as-Treated and Evaluable populations, and comparisons were made across treatment groups as described in Table 11. Additional subgroup analyses were also performed on any prognostic factors identified in the analysis of 28-day (672-hour post first dose of study drug) all-cause mortality.

### Secondary Efficacy Endpoint Analysis Results

No statistically significant differences were noted between treatment groups (TLF and placebo) in either the ITT-as-Treated or Evaluable populations for the following endpoints: the number of ICU days for survivors, the proportion of shock-free days in the ICU, the proportion of ventilator-free days in the ICU, the proportion of dialysis-free days in the ICU, the proportion of organ dysfunction-free days in the ICU, the duration of use of vasopressor medications in the ICU, and the incidence and severity of additional organ failure/dysfunction in the ICU. The data are not normally distributed; therefore, non-parametric techniques such as the Wilcoxon Rank Sum test were used in the analyses.

TABLE 7
Analysis of Secondary Efficacy Endpoints (ITT-as-Treated and Evaluable Populations)

| | ITT-as-Treated Population | | | Evaluable Population | | |
|---|---|---|---|---|---|---|
| | Treatment Group | | | Treatment Group | | |
| | TLF | Placebo | P | TLF | Placebo | P |
| Secondary Efficacy Endpoint | N = 96 | N = 94 | value^{a} | N = 90 | N = 85 | value^{a} |
| Number of ICU days for survivors (N) | 87 | 79 | 0.3520 | 86 | 77 | 0.3294 |
| Mean ± SD | 9.0 ± 9.31 | 7.7 ± 8.60 | | 9.0 ± 9.37 | 7.6 ± 8.55 | |
| Median (Min, Max) | 6.0 (1, 41) | 5.0 (1, 48) | | 6.0 (1, 41) | 5.0 (1, 48) | |
| Proportion of shock-free days in the ICU (N) | 96 | 94 | 0.4471 | 90 | 85 | 0.6849 |
| Mean ± SD | 0.51 ± 0.31 | 0.48 ± 0.31 | | 0.55 ± 0.29 | 0.53 ± 0.29 | |
| Median (Min, Max) | 0.50 (0.0, 1.0) | 0.50 (0.0, 1.0) | | 0.58 (0.0, 1.0) | 0.50 (0.0, 1.0) | |
| Proportion of ventilator-free days in the ICU (N) | 96 | 94 | 0.8955 | 90 | 85 | 0.8129 |
| Mean ± SD | 0.45 ± 0.33 | 0.44 ± 0.33 | | 0.47 ± 0.33 | 0.48 ± 0.31 | |
| Median (Min, Max) | 0.50 (0.0, 1.0) | 0.42 (0.0, 1.0) | | 0.50 (0.0, 1.0) | 0.50 (0.0, 1.0) | |
| Proportion of dialysis-free days in the ICU^{b} (N) | 94 | 92 | 0.3569 | 88 | 83 | 0.4869 |
| Mean ± SD | 0.83 ± 0.18 | 0.84 ± 0.22 | | 0.83 ± 0.18 | 0.83 ± 0.21 | |
| Median (Min, Max) | 0.88 (0.0, 1.0) | 0.92 (0.0, 1.0) | | 0.87 (0.0, 1.0) | 0.89 (0.0, 1.0) | |
| Proportion of organ dysfunction-free days in the | 96 | 94 | 0.2664 | 90 | 85 | 0.3942 |
| ICU (N) | | | | | | |
| Mean ± SD | 0.23 ± 0.26 | 0.20 ± 0.25 | | 0.25 ± 0.26 | 0.22 ± 0.26 | |
| Median (Min, Max) | 0.14 (0.0, 0.9) | 0.00 (0.0, 0.8) | | 0.17 (0.0, 0.9) | 0.13 (0.0, 0.8) | |
| Number of days on vasopressor medications in the | 96 | 94 | 0.9088 | 90 | 85 | 0.9951 |
| ICU (N) | | | | | | |
| Mean ± SD | 2.2 ± 2.52 | 2.3 ± 3.10 | | 2.2 ± 2.60 | 2.3 ± 3.25 | |
| Median (Min, Max) | 2.0 (0, 12) | 2.0 (0, 21) | | 2.0 (0, 12) | 2.0 (0, 21) | |

Reference Table 14.2.3.1-2, 14.2.4.1-2, 14.2.5.1-2, 14.2.6.1-2, 14.2.7.1-2, and 14.2.8.1-2
^{a}P-values based on a 2-sided Wilcoxon Rank Sum test
^{b}Both the ITT-as-Treated and Evaluable analyses exclude those patients who were receiving dialysis prior to being diagnosed with sepsis.

Additional secondary analyses to determine 3-month and 6-month all-cause mortality and time to death were conducted for the ITT-as-Treated and the Evaluable populations.

In the ITT-as-Treated population overall, the ACM appeared to remain fairly constant between 3 and 6 months post treatment for subjects treated with TLF. Similar results were observed in the Evaluable population.

TABLE 8
Analysis of 3- and 6-Month All-Cause Mortality Stratified by Cardiovascular Dysfunction (ITT-as-Treated and Evaluable Populations)

| | 3-Month Mortality | | | | | | 6-Month Mortality | | | | | |
|---|---|---|---|---|---|---|---|---|---|---|---|---|
| | ITT-as-Treated Population | | | Evaluable Population | | | ITT-as-Treated Population | | | Evaluable Population | | |
| | Treatment Group | | | Treatment Group | | | Treatment Group | | | Treatment Group | | |
| | TLF | Placebo | | TLF | Placebo | | TLF | Placebo | | TLF | Placebo | |
| Primary | N = 96 | N = 94 | P | N = 90 | N = 85 | P | N = 96 | N = 94 | P | N = 90 | N = 85 | P |
| Endpoint | n (%) | n (%) | value^{a,b} | n (%) | n (%) | value^{a,b} | n (%) | n (%) | value^{a,b} | n (%) | n (%) | value^{a,b} |
| Overall | | | | | | | | | | | | |
| Population | | | | | | | | | | | | |
| Alive | 74 (82.2) | 64 (71.9) | 0.1514^{a} | 74 (87.1.) | 63 (78.8) | 0.2441^{a} | 74 (79.6) | 59 (66.3) | 0.0570^{a} | 74 (84.1) | 59 (73.8) | 0.1306^{a} |
| | | | 0.1755^{b} | | | 0.7008^{b} | | | 0.4325^{b} | | | 0.9481^{b} |
| Deceased | 16 (17.8) | 25 (28.1) | | 11 (12.9) | 17 (21.3) | | 19 (20.4) | 30 (33.7) | | 14 (15.9) | 21 (26.3) | |
| Lost to | 6 | 5 | | 5 | 5 | | 3 | 5 | | 2 | 3 | |
| follow-up | | | | | | | | | | | | |
| Cardiovascular | | | | | | | | | | | | |
| Dysfunction | | | | | | | | | | | | |
| Present at | | | | | | | | | | | | |
| Baseline | | | | | | | | | | | | |
| Alive | 39 (73.6) | 41 (68.3) | | 39 (81.3) | 40 (74.1) | | 41 (73.2) | 37 (62.7) | | 41 (80.4) | 37 (69.8) | |
| Deceased | 16 (17.8) | 25 (28.1) | | 9 (18.8) | 2 (5.4) | | 15 (26.8) | 22 (37.3) | | 10 (19.6) | 16 (30.2) | |
| Lost to | 5 | 3 | | 4 | 3 | | 2 | 4 | | 1 | 4 | |
| Follow-Up | | | | | | | | | | | | |
| Absent at | | | | | | | | | | | | |
| Baseline | | | | | | | | | | | | |
| Alive | 35 (94.6) | 23 (79.3) | | 35 (94.6) | 23 (88.5) | | 33 (89.2) | 22 (73.3) | | 33 (89.2) | 22 (81.5) | |
| Deceased | 2 (5.4) | 6 (20.7) | | 2 (5.4) | 3 (11.5) | | 4 (10.8) | 8 (26.7) | | 4 (10.8) | 5 (18.5) | |
| Lost to | 1 | 2 | | 1 | 2 | | 1 | 1 | | 1 | 1 | |
| Follow-Up | | | | | | | | | | | | |

Reference Tables 14.2.12.1-2, 14.2.13.1-2
^{a}P-value from Two-Sided Cochran-Mantel-Haenszel Test
^{b}P-value from Two-sided Breslow-Day Test

The number of subjects who have died while on study was insufficient to estimate median survival times from Kaplan-Meier analyses. Instead, the number of deaths and the time to death were tabulated for each treatment group and analyzed for both the ITT-as-Treated and the Evaluable populations. The results illustrate a relatively small number of deaths, a wide range of values for time to death, and no significant difference between treatment groups in the time to death post-treatment.

TABLE 9
Summary of Time to Death for Deceased Patients by
Treatment Group (ITT-as-Treated Population)

Treatment Group

| | TLF | Placebo | |
|---|---|---|---|
| Population Evaluated^{a} | N | N | P value^{b} |
| ITT-as-Treated | N = 96 | N = 94 | |
| Number Deceased | 19 | 30 | |
| Number of Days | | | |
| Mean ± SD | 33.7 ± 43.1 | 34.0 ± 49.2 | 0.9918 |
| Median (Min, Max) | 12.3 (0, 135) | 13.3 (0, 164) | |
| Evaluable Population | N = 90 | N = 85 | |
| Number Deceased | 14 | 21 | |
| Number of Days | | | |
| Mean ± SD | 45.5 ± 44.8 | 41.1 ± 50.7 | 0.6254 |
| Median (Min, Max) | 24.5 (2, 135) | 14.3 (2, 164) | |

Reference: Tables 14.2.14.3.1, 14.2.14.3.2
^{a}Three subjects who withdrew prior to the 672-hour survival endpoint were imputed as deceased due to having organ dysfunction at the time of last contact.
^{b}p-Value from Two-sided Wilcoxon Rank Sum Test

ii. Statistical/Analytical Issues

No statistical issues were observed that would result in the inappropriate use of statistical methods used to analyze the data.

Patients with APACHE Scores greater than 25 at baseline demonstrated a significantly lower ACM following treatment with TLF.

More extensive data are available with regard to the effects of organ dysfunction on TLF. As noted earlier, no statistically significant differences between treatment groups are noted at baseline in the type of organ dysfunction by number of organs with dysfunction (Table 17, and Tables 14.2.17 and 14.2.18). The data below also suggest that the presence of metabolic dysfunction with or without CV dysfunction, or the presence of ≦2 organs with dysfunction, may influence the effects of TLF, although the differences are not statistically significant.

TABLE 10
Mortality by Number and Type of Organ
Dysfunctions at Baseline and by Treatment Group
(ITT-as-Treated Population)

| | Treatment Group | | | |
|---|---|---|---|---|
| | TLF | | Placebo | |
| | N = 96 | | N = 94 | |
| Organ Dysfunction | n | (%) | n | (%) |
| Type of Organ Dysfunction by Number | | | | |
| of Organs with Dysfunction | | | | |
| 1 Organ | | | | |
| Cardiovascular | 14 | (31.1) | 14 | (42.4) |
| Respiratory | 19 | (42.2) | 15 | (45.5) |
| Renal | 5 | (11.1) | 2 | (6.1) |
| Hematologic | 5 | (11.1) | 1 | (3.0) |
| Metabolic | 2 | (4.4) | 1 | (3.0) |
| 2 Organs | | | | |
| Cardiovascular | 18 | (72.0) | 21 | (65.6) |
| Respiratory | 12 | (48.0) | 19 | (59.4) |
| Renal | 9 | (36.0) | 13 | (40.6) |
| Hematologic | 4 | (16.0) | 3 | (9.4) |
| Metabolic | 7 | (28.0) | 8 | (25.0) |
| 3 Organs | | | | |
| Cardiovascular | 18 | (100.0) | 17 | (100.0) |
| Respiratory | 12 | (66.7) | 11 | (64.7) |
| Renal | 12 | (66.7) | 10 | (58.8) |
| Hematologic | 3 | (16.7) | 2 | (11.8) |
| Metabolic | 9 | (50.0) | 11 | (64.7) |
| 4 Organs | | | | |
| Cardiovascular | 6 | (100.0) | 9 | (90.0) |
| Respiratory | 6 | (100.0) | 10 | (100.0) |
| Renal | 6 | (100.0) | 9 | (90.0) |
| Hematologic | 0 | (0.0) | 3 | (30.0) |
| Metabolic | 6 | (100.0) | 9 | (90.0) |
| 5 Organs | | | | |
| Cardiovascular | 2 | (100.0) | 2 | (100.0) |
| Respiratory | 2 | (100.0) | 2 | (100.0) |
| Renal | 2 | (100.0) | 2 | (100.0) |
| Hematologic | 2 | (100.0) | 2 | (100.0) |
| Metabolic | 2 | (100.0) | 2 | (100.0) |
| 28-Day Mortality by Type of Organ | | | | |
| Dysfunction | | | | |
| Cardiovascular | | | | |
| Alive | 45 | (77.6) | 45 | (71.4) |
| Deceased | 13 | (22.4) | 18 | (28.6) |
| Respiratory | | | | |
| Alive | 44 | (86.3) | 40 | (70.2) |
| Deceased | 7 | (13.7) | 18 | (28.6) |
| Renal | | | | |
| Alive | 27 | (79.4) | 23 | (63.9) |
| Deceased | 7 | (13.7) | 17 | (29.8) |
| Hematologic | | | | |
| Alive | 12 | (85.7) | 7 | (63.6) |
| Deceased | 2 | (14.3) | 4 | (36.4) |
| Metabolic | | | | |
| Alive | 17 | (65.4) | 21 | (67.7) |
| Deceased | 9 | (34.6) | 10 | (32.3) |
| Cardiovascular + Metabolic | | | | |
| Alive | 12 | (60.0) | 18 | (72.0) |
| Deceased | 8 | (40.0) | 7 | (28.0) |
| Cardiovascular + Respiratory | | | | |
| Alive | 21 | (75.0) | 21 | (65.6) |
| Deceased | 7 | (25.0) | 11 | (34.4) |
| 28-Day Mortality by Number of | | | | |
| Organs with Dysfunction | | | | |
| 1 Organ | | | | |
| Alive | 42 | (93.3) | 29 | (87.9) |
| Deceased | 3 | (6.7) | 4 | (12.1) |
| 2 Organs | | | | |
| Alive | 23 | (92.0) | 21 | (65.6) |
| Deceased | 2 | (8.0) | 11 | (34.4) |
| 3 Organs | | | | |
| Alive | 13 | (72.2) | 12 | (70.6) |
| Deceased | 5 | (27.8) | 5 | (29.4) |
| 4 Organs | | | | |
| Alive | 2 | (33.3) | 6 | (60.0) |
| Deceased | 4 | (66.7) | 4 | (40.0) |
| 5 Organs | | | | |
| Alive | 2 | (100.0) | 1 | (50.0) |
| Deceased | 0 | (0.0) | 1 | (50.0) |

Reference: Table 14.2.18
^{a}Three subjects who withdrew prior to the 672-hour survival endpoint were imputed as deceased due to having organ dysfunction at the time of last contact.
^{b}Patients are counted in more than 1 type for the categories “More than one organ with dysfunction.

### Efficacy Summary

Analyses were performed in the most conservative fashion possible, taking into account the dosing and randomization errors that occurred in 44 patients during the study. Treatment groups appear similar in terms of baseline characteristics measured.
Primary endpoint: analyzed by logistic regression (in all 4 efficacy populations) and by Kaplan-Meier analysis (in the ITT as treated and Evaluable population), a 45% reduction in 28 day all-cause mortality is noted in the TLF group when compared to the placebo group. The data did not allow calculate median time to death statistics, but overall Kaplan-Meier results are consistent with those of logistic regression. Results in the evaluable population were similar.
Sensitivity analyses were performed on 168 subjects (ITT-as-Treated population), which excluded those 22 subjects who received both placebo and TLF due to the vial labeling error. The results of the sensitivity analyses show no differences in the conclusions when these 22 subjects are included in analyses
When data were analyzed for subjects with CV dysfunction, a relatively small but notable difference between the treatment groups was observed. Compared to results with Xigris and in the Eritoran trails that have progressed into Phase III or been approved, the absolute reduction seen in this Phase II trial is consistent with the level of activity reported with those agents. The PIs felt that an absolute reduction of 6% in mortality was clinically significant—
Secondary endpoints: No statistical significance was determined between treatment groups for most of these exploratory endpoints.
Determination of 3 and 6 month ACM and time-to-death tabulations for treatment groups revealed a higher number of deaths overall in groups treated with placebo for both the ITT-as-Treated and the Evaluable populations, but no statistically significant difference between treatment groups was noted in the mean time to death (in days).
The presence of metabolic dysfunction with or without CV dysfunction, or the presence of ≦2 organs with dysfunction, may have some influence on the effects of TLF, although the differences observed in this study were not statistically significant.
Patients with APACHE Scores greater than 25 at baseline demonstrated a significantly lower ACM following treatment with TLF.
Several baseline characteristics may be considered to be potential prognostic factors, and further investigation into some of these may be warranted, especially APACHE scores, metabolic vs. non-metabolic organ dysfunction, the number of organ dysfunctions, and gender.
This study provides strong evidence of the efficacy of TLF in the treatment of severe sepsis. TLF is associated with lower mortality rates in patients with severe sepsis overall and in those subjects with no CV dysfunction, when compared to placebo.

### Safety Evaluation

### Analysis of Safety

All subjects who received at least 1 dose of TLF were included in the TLF group for safety analyses. The Safety Populations consisted of those subjects who met the criteria outlined below:

Safety Population Group A
If the subject received TLF first, the subject was included in the TLF treatment group only and all safety data for that subject were attributed to TLF. If the subject received placebo first, safety data for that subject were included in the placebo group up until the time the subject received TLF, at which point all safety data for that subject were attributed to TLF for the remainder of the study. This was the primary method utilized for analyses using the safety population. This population is comprised of 190 subjects (TLF N=109, placebo N=94).

Safety Population B
Include the subject in the TLF treatment group only. This population consisted of TLF N=109 and placebo N=81.
For the safety analysis, safety data recorded while the 22 subjects were on the incorrect treatment were summarized by the treatment group in which those subjects received the majority of treatment. AE data were listed separately to investigate any clinically significant changes as a result of treatment. Analyses of the Safety Population Group B revealed no differences from Safety Population Group A.

TABLE 11
Summary of Safety Analyses

| Measurement | Endpoint | Analysis |
|---|---|---|
| Adverse events | Summary of treatment | Tabulations and |
| | emergent adverse events | listings |
| | | Fisher's exact test |
| | | Two-sample t-test |
| Laboratory | Percentage change from | Summary statistics |
| assessment | baseline in laboratory | Paired t-test |
| | values and transition | Two-sample t-test |
| | tables from baseline in | McNemar's/Bowker's |
| | CTC grade and clinically | test |
| | significant values | |
| Vital signs | Percentage change from | Summary statistics |
| | baseline in vital signs | Paired t-test |
| | | Two-sample t-test |
| Physical Exam | Transition table from | Tabulations and |
| | baseline | listings |
| Concomitant | Summary of | Tabulations and |
| medications | concomitant | listings |
| | medications | |
| Cytokines | Percentage change from | Summary statistics |
| | baseline in cytokines | Paired t-test |
| | | Two-sample t-test |

### Safety Summary

TLF was well-tolerated.
   No clinically or statistically significant differences were noted between treatment groups within the severity categories of Mild, Moderate and Severe.
   No statistically significant differences were noted between treatment groups within the severity categories of Grade 3, 4, and 5; however, a slightly higher number and percentage of AEs were reported in the TLF group, attributed to longer survival in the TLF-treated subjects.
   No definitely-related AEs were reported.
   GI effects (AEs) were equally distributed between treatment groups. The most frequently reported treatment-related AEs in both treatment groups were GI disorders, accounting for the majority of possibly-related AEs in both treatment groups.
   Borderline statistically significant AE p-values were noted in the incidence and percentage of Hyperkalemia, Fluid Overload, Metabolic Encephalopathy, Pneumothorax, and Skin and Subcutaneous Disorders. A statistically significant difference was noted in Atrial Fibrillation, perhaps warrants further exploration in future studies.
   No statistically significant differences were noted between treatment groups in Vital signs or Physical examinations
DSMB ascertained no safety issues arose due to vial labeling dosing errors, therefore the study proceeded with enrollment as planned.
All AEs were deemed as Unrelated, Unlikely or Possibly-related to study treatment. No Probably or Definitely-related AEs were reported.
All SAEs in the TLF or placebo groups were deemed as Unrelated or Unlikely-related to study treatment. No SAEs were deemed as Possibly, Probably or Definitely-related to study treatment. SAEs were equally distributed between treatment groups with no clinically significant differences noted. No SAEs required expedited reporting to the FDA.
Deaths—19 deaths were attributed to TLF, 30 attributed to placebo-2 of the TLF subjects received both treatment drugs and 5 of the placebo subjects received both treatment drugs due to vial mislabeling dosing errors.
No statistically significant differences between treatment groups were noted in Grade 3 elevation laboratory values with the exception of hemoglobin, platelet count and bicarbonate, which perhaps warrants further examination in future studies. Difficult to ascertain C-reactive protein results due to wide range of values.

### Analysis of Adverse Events

Analysis of the AEs reported for this study indicates that TLF was well-tolerated. The AE profile of TLF is similar to that of Placebo. No clinical or statistically significant differences are noted between treatment groups in the incidence of AEs in the Mild, Moderate and Severe categories.
When AEs of Grade 3, 4, and 5 are grouped together, the TLF-treated subjects demonstrate a slightly higher incidence of AEs when compared to placebo: 153 AEs for TLF (53.7% of AEs reported in Grade 3, 4, and 5) and 132 AEs for placebo (46.3% of AEs reported in Grade 3, 4, and 5). This difference is statistically insignificant (p-value=0.2135), and may be attributed to longer survival in the TLF-treated subjects. Please refer to the End-of-Text Table 14.3.1.11.2)
Thirty-five percent of the 1033 AEs reported were Mild, 37% were Moderate in intensity, and 19% were Severe. A total of 198 severe AEs were reported as follows: 108 in the TLF group and 90 in the placebo group. Please refer to Table X above and End-of-Text Tables 14.3.X.X
All AEs in this study were deemed as either Unrelated, Unlikely or Possibly Related to study treatment; none were reported as Probably or Definitely Related to study treatment. GI disorders accounted for the majority of Possibly-Related AEs in both the TLF and placebo groups, which included 3 Possibly-Related Diarrhea AEs (2.8%) attributed to TLF (all 3 deemed as Mild) and 3 Possibly-Related Vomiting AEs (3.2%) attributed to placebo (1 deemed as Moderate and 1 deemed as Severe). Please refer to the End-of-Text Table 14.3.1.6.1.

### Deaths, Other Serious, and Other Significant Adverse Events

### Listing of Deaths, Other Serious, and Other Significant Adverse Events

Deaths
Forty-nine deaths occurred during this study; 19 were attributed to TLF and 30 were attributed to placebo. Seven of the deceased subjects received both study treatments (2 patients in the TLF group and 5 patients in the Placebo group [ITT as Treated]) due to vial mislabeling dosing errors.

Positive Results with Talactoferrin In a Randomized, Double-Blind, Placebo-Controlled Phase 2 Trial in Severe Sepsis were obtained.
Trial results show 45% overall reduction in 28-day all cause mortality with talactoferrin versus placebo
Talactoferrin again shown to be very well tolerated

Results from the talactoferrin randomized, double-blind, placebo-controlled Phase 2 trial in severe sepsis. The trial evaluated talactoferrin versus placebo in 190 adult patients with severe sepsis enrolled at 25 leading centers across the U.S. Patients in both arms also received standard of care treatment for severe sepsis in an intensive care unit (ICU) setting. The trial achieved its primary endpoint of a reduction in 28-day all-cause mortality. The trial showed a 45% reduction in the 28-day all-cause mortality from 26.6% in the placebo arm to 14.6% in the talactoferrin arm (two-tailed p-value=0.04, odds ratio by logistic regression analysis=0.47).

Patients were stratified by clinical site and by the presence or absence of cardiovascular dysfunction. Cardiovascular dysfunction is a major prognostic factor for severe sepsis. A similar number of patients had cardiovascular dysfunction in the two treatment groups. Sixty-four percent (64%) of patients (n=121) in the trial had cardiovascular dysfunction and 36% (n=69) did not. For those patients with cardiovascular dysfunction, 28-day all cause mortality was 28.6% for the placebo arm and 22.4% for the talactoferrin arm. For patients who did not have cardiovascular dysfunction, 28-day all cause mortality was 22.6% in the placebo arm compared to 2.6% in the talactoferrin arm. When the trial results were adjusted for cardiovascular dysfunction, the two-tailed p-value was 0.06, and the odds ratio was 0.49.

The above analyses were all conducted on an intent-to-treat (ITT), as treated basis, meaning that patients were evaluated based on the treatment they actually received (talactoferrin or placebo). An ITT as treated analysis is a method to address patient assignment errors in a way that mitigates the potential impact on the data analysis of a trial. In this study, the quality control process identified errors in drug labeling and randomization during the conduct of the trial that affected the drug assignment for some patients. That is why this analysis was used, following feedback from the U.S. Food and Drug Administration (FDA). To determine if the assignment error had an impact on the outcome of the trial, as recommended by the FDA, the Company conducted a sensitivity analysis evaluating 28-day all cause mortality by excluding 22 patients who mistakenly received both talactoferrin and placebo. This analysis indicated that there was no apparent effect of the patient assignment errors on the outcome of the trial. The sensitivity analysis showed that 28-day all cause mortality in the placebo arm was 25.9% compared to 15.1% for the talactoferrin arm.

Talactoferrin was shown to be very well tolerated in the study with no major differences in adverse events between the two treatment arms.

The study included 96 patients in the talactoferrin arm and 94 patients in the placebo arm. In addition, four patients were randomized but did not receive study drug due to withdrawal prior to receiving the first dose. All patients were centrally screened for eligibility prior to randomization. The arms were well balanced in terms of baseline characteristics.

1.-10. (canceled)

11. A method for treating sepsis in a patient comprising administering to the patient having severe sepsis a composition comprising lactoferrin in a pharmaceutically acceptable carrier.

12. The method of claim 11, wherein the composition is administered orally.

13. The method of claim 11, wherein the patient has a baseline APACHE II score of ≦25.

14. The method of claim 11, wherein the patient has a baseline APACHE II score of <21.

15. The method of claim 11, wherein the severe sepsis comprises at least one organ dysfunction.

16. The method of claim 11, wherein the severe sepsis comprises no more than one organ dysfunction.

17. The method of claim 11, wherein the patient has unimpaired cardiovascular function.

18. The method of claim 11, wherein the lactoferrin is human lactoferrin.

19. The method of claim 11, wherein the patient is less than 18 years old.

20. The method of claim 11, wherein the lactoferrin in the composition is administered in a dosage amount of 1.5 mg to 100 g every 8 hours.

21. The method of claim 11, wherein the lactoferrin in the composition is administered in a dosage amount of 1.0 mg to 5 g every 8 hours.

22. The method of claim 12, wherein the lactoferrin in the composition is administered orally and the lactoferrin in the composition is in a dosage amount of 1.5 mg to 100 g every 8 hours.

23. The method of claim 12, wherein the lactoferrin in the composition is administered orally and the lactoferrin in the composition is in a dosage amount of 1.0 mg to 5 g every 8 hours.

24. The method of claim 11, wherein the treatment of severe sepsis in the patient is therapeutic.

25. The method of claim 11, wherein the treatment of severe sepsis in the patient is prophylactic.
